SEQUENCE LISTING (19 sequences)

SEQ ID NO: 1 - protein, 529 aa, Artificial Sequence
  Spastin
MAAKRSSGTAPAPASPSPPAPGPGGEAESVRVFHKQAFEYISIALRIDEEEKGQKEQAVEWYKKGIEELEKGIAVIVTGQGEQYERARRLQAKMMTNLVMAKDRLQLLEKLQPVLQFSKSQTDVYNESTNLTCRNGHLQSESGAVPKRKDPLTHASNSLPRSKTVLKSGSAGLSGHHRAPSCSGLSMVSGARPGPGPAATTHKGTPKPNRTNKPSTPTTAVRKKKDLKNFRNVDSNLANLIMNEIVDNGTAVKFDDIAGQELAKQALQEIVILPSLRPELFTGLRAPARGLLLFGPPGNGKTMLAKAVAAESNATFFNISAASLTSKYVGEGEKLVRALFAVARELQPSIIFIDEVDSLLCERREGEHDASRRLKTEFLIEFDGVQSAGDDRVLVMGATNRPQELDEAVLRRFIKRVYVSLPNEETRLLLLKNLLCKQGSPLTQKELAQLARMTDGYSGSDLTALAKDAALGPIRELKPEQVKNMSASEMRNIRLSDFTESLKKIKRSVSPQTLEAYIRWNKDFGDTTV

SEQ ID NO: 2 - protein, 390 aa, Artificial Sequence
  dNSpastin
MESGAVPKRKDPLTHASNSLPRSKTVLKSGSAGLSGHHRAPSCSGLSMVSGARPGPGPAATTHKGTPKPNRTNKPSTPTTAVRKKKDLKNFRNVDSNLANLIMNEIVDNGTAVKFDDIAGQELAKQALQEIVILPSLRPELFTGLRAPARGLLLFGPPGNGKTMLAKAVAAESNATFFNISAASLTSKYVGEGEKLVRALFAVARELQPSIIFIDEVDSLLCERREGEHDASRRLKTEFLIEFDGVQSAGDDRVLVMGATNRPQELDEAVLRRFIKRVYVSLPNEETRLLLLKNLLCKQGSPLTQKELAQLARMTDGYSGSDLTALAKDAALGPIRELKPEQVKNMSASEMRNIRLSDFTESLKKIKRSVSPQTLEAYIRWNKDFGDTTV

SEQ ID NO: 3 - protein, 529 aa, Artificial Sequence
  SpastinED
MAAKRSSGTAPAPASPSPPAPGPGGEAESVRVFHKQAFEYISIALRIDEEEKGQKEQAVEWYKKGIEELEKGIAVIVTGQGEQYERARRLQAKMMTNLVMAKDRLQLLEKLQPVLQFSKSQTDVYNESTNLTCRNGHLQSESGAVPKRKDPLTHASNSLPRSKTVLKSGSAGLSGHHRAPSCSGLSMVSGARPGPGPAATTHKGTPKPNRTNKPSTPTTAVRKKKDLKNFRNVDSNLANLIMNEIVDNGTAVKFDDIAGQELAKQALQEIVILPSLRPELFTGLRAPARGLLLFGPPGNGKTMLAKAVAAESNATFFNISAASLTSKYVGEGEKLVRALFAVARELQPSIIFIDAVDSLLCERREGEHDASRRLKTEFLIEFDGVQSAGDDRVLVMGATNRPQELDEAVLRRFIKRVYVSLPNEETRLLLLKNLLCKQGSPLTQKELAQLARMTDGYSGSDLTALAKDAALGPIRELKPEQVKNMSASEMRNIRLSDFTESLKKIKRSVSPQTLEAYIRWNKDFGDTTV

SEQ ID NO: 4 - protein, 390 aa, Artificial Sequence
  dNSpastinED
MESGAVPKRKDPLTHASNSLPRSKTVLKSGSAGLSGHHRAPSCSGLSMVSGARPGPGPAATTHKGTPKPNRTNKPSTPTTAVRKKKDLKNFRNVDSNLANLIMNEIVDNGTAVKFDDIAGQELAKQALQEIVILPSLRPELFTGLRAPARGLLLFGPPGNGKTMLAKAVAAESNATFFNISAASLTSKYVGEGEKLVRALFAVARELQPSIIFIDAVDSLLCERREGEHDASRRLKTEFLIEFDGVQSAGDDRVLVMGATNRPQELDEAVLRRFIKRVYVSLPNEETRLLLLKNLLCKQGSPLTQKELAQLARMTDGYSGSDLTALAKDAALGPIRELKPEQVKNMSASEMRNIRLSDFTESLKKIKRSVSPQTLEAYIRWNKDFGDTTV

SEQ ID NO: 5 - protein, 286 aa, Artificial Sequence
  dNSpastinCD
MIVDNGTAVKFDDIAGQELAKQALQEIVILPSLRPELFTGLRAPARGLLLFGPPGNGKTMLAKAVAAESNATFFNISAASLTSKYVGEGEKLVRALFAVARELQPSIIFIDEVDSLLCERREGEHDASRRLKTEFLIEFDGVQSAGDDRVLVMGATNRPQELDEAVLRRFIKRVYVSLPNEETRLLLLKNLLCKQGSPLTQKELAQLARMTDGYSGSDLTALAKDAALGPIRELKPEQVKNMSASEMRNIRLSDFTESLKKIKRSVSPQTLEAYIRWNKDFGDTTV

SEQ ID NO: 6 - protein, 390 aa, Artificial Sequence
  dNSpastin3Q
MESGAVPKRKDPLTHASNSLPRSKTVLKSGSAGLSGHHRAPSCSGLSMVSGARPGPGPAATTHKGTPKPNRTNKPSTPTTAVRQQQDLKNFRNVDSNLANLIMNEIVDNGTAVKFDDIAGQELAKQALQEIVILPSLRPELFTGLRAPARGLLLFGPPGNGKTMLAKAVAAESNATFFNISAASLTSKYVGEGEKLVRALFAVARELQPSIIFIDEVDSLLCERREGEHDASRRLKTEFLIEFDGVQSAGDDRVLVMGATNRPQELDEAVLRRFIKRVYVSLPNEETRLLLLKNLLCKQGSPLTQKELAQLARMTDGYSGSDLTALAKDAALGPIRELKPEQVKNMSASEMRNIRLSDFTESLKKIKRSVSPQTLEAYIRWNKDFGDTTV

SEQ ID NO: 7 - protein, 390 aa, Artificial Sequence
  dNSpastin3QED
MESGAVPKRKDPLTHASNSLPRSKTVLKSGSAGLSGHHRAPSCSGLSMVSGARPGPGPAATTHKGTPKPNRTNKPSTPTTAVRQQQDLKNFRNVDSNLANLIMNEIVDNGTAVKFDDIAGQELAKQALQEIVILPSLRPELFTGLRAPARGLLLFGPPGNGKTMLAKAVAAESNATFFNISAASLTSKYVGEGEKLVRALFAVARELQPSIIFIDAVDSLLCERREGEHDASRRLKTEFLIEFDGVQSAGDDRVLVMGATNRPQELDEAVLRRFIKRVYVSLPNEETRLLLLKNLLCKQGSPLTQKELAQLARMTDGYSGSDLTALAKDAALGPIRELKPEQVKNMSASEMRNIRLSDFTESLKKIKRSVSPQTLEAYIRWNKDFGDTTV

SEQ ID NO: 8 - protein, 158 aa, Artificial Sequence
  C2Lact
CANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 9 - protein, 282 aa, Artificial Sequence
  EMTB
MEQKLISEEDLGAPVRSETAPDSYKVQDKKNASSRPASAISGQNNNHSGNKPDPPPVLRVDDRQRLARERREEREKQLAAREIVWLEREERARQHYEKHLEERKKRLEEQRQKEERRRAAVEEKRRQRLEEDKERHEAVVRRTMERSQKPKQKHNRWSWGGSLHGSPSIHSADPDRRSVSTMNLSKYVDPVISKRLSSSSATLLNSPDRARRLQLSPWESSVVNRLLTPTHSFLARSKSTAALSGEAASCSPIIMPYKAAHSRNSMDRPKLFVTPPEGSSDP

SEQ ID NO: 10 - protein, 107 aa, Artificial Sequence
  FKBP
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 11 - protein, 93 aa, Artificial Sequence
  FRB
ILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 12 - protein, 92 aa, Artificial Sequence
  GAIs
MKRDHHHHHHQDKKTMMMNEEDDGNGMDELLAVLGYKVRSSEMADVAQKLEQLEVMMSNVQEDDLSQLATETVHYNPAELYTWLDSMLTDLN

SEQ ID NO: 13 - protein, 345 aa, Artificial Sequence
  mGID1
MAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAEC

SEQ ID NO: 14 - protein, 498 aa, Artificial Sequence
  Cry2
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELRATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAA

SEQ ID NO: 15 - protein, 82 aa, Artificial Sequence
  CIBN
MNGAIGGDLLLNFPDMSVLERQRAHLKYLNPTFDSPLAGFFADSSMITGGEMDSYLSTAGLNLPMMYGETTVEGDSRLSISV

SEQ ID NO: 16 - protein, 64 aa, Artificial Sequence
  A1AY1
MATVKFKYKGEEKQVDISKILSVGRYGKLIHFLYDLGGGKAGMGMVSEKDAPKELLQMLEKQKK

SEQ ID NO: 17 - protein, 390 aa, Artificial Sequence; identical to SEQ ID NO: 2
  Isoform of Spastin

SEQ ID NO: 18 - protein, 240 aa, Artificial Sequence
  TagRFP
MVSKGEELIKENMHMKLYMEGTVNNHHFKCTSEGEGKPYEGTQTMRIKVVEGGPLPFAFDILATSFMYGSRTFINHTQGIPDFFKQSFPEGFTWERVTTYEDGGVLTATQDTSLQDGCLIYNVKIRGVNFPSNGPVMQKKTLGWEANTEMLYPADGGLEGRTDMALKLVGGGHLICNFKTTYRSKKPAKNLKMPSVYYVDHRLERIKEADKETYVEQHEVAVARYCDLPSKLGHKLNLYK

SEQ ID NO: 19 - protein, 317 aa, Artificial Sequence
  MAP4m
MSGSKSTQTVAKTTTAAAVASTGPSSRSPSTLLPKKPTAIKTEGKPAEVKKMTAKSVPADLSRPKSTSTSSMKKTTTLSGTAPAAGVVPSRVKATPMPSRPSTTPFIDKKPTSAKPSSTTPRLSRLATNTSAPDLKNVRSKVGSTENIKHQPGGGRVQIVSKKVSYSHIQSKCGSKDNIKHVPGGGNVQIQNKKVDISKVSSKCGSKANIKHKPGGGDVKIESQKLNFKEKAQAKVGSLDNVGHLPAGGAVKTEGGGSEAPLCPGPPAGEEPAISEAAPEAGAPTSASGLNGHPTLSGGGDQREAQTLDSQIQETSI

(19) United States
(12) Patent Application Publication
(10) Pub. No.: US 2023/0203471 A1
(43) Pub. Date: Jun. 29, 2023

(54) METHOD AND PLATFORM FOR DISRUPTING INTRACELLULAR MICROTUBULES

(71) Applicant: National Tsing Hua University, Hsinchu (TW)

(72) Inventors: Yu-Chun Lin, Hsinchu (TW); Grace Y. Liu, Hsinchu (TW)

(21) Appl. No.: 17/735,595

(22) Filed: May 3, 2022

(30) Foreign Application Priority Data
Dec. 24, 2021 (TW) 110148789

(51) Int. Cl. C12N 13/00; C12N 9/90; C07K 14/47

(52) U.S. Cl. CPC C12N 13/00; C07K 14/47; C12N 9/90; C12Y 502/01008; C12N 2501/70; C12N 2501/999; C12N 2529/10

(57) ABSTRACT

The present disclosure provides a method and a platform for disrupting intracellular microtubules. The method and the platform of the present disclosure can accurately and quickly disrupt the microtubule structure in a specific area of a cell by adding a specific chemical small molecule or a specific wavelength of light. In addition to providing important reagents for microtubule-related research in basic science, it may even be developed into a new technology for precise chemotherapy.

### CROSS REFERENCE TO RELATED APPLICATION

This application claims priority of Taiwan patent application No. 110148789, filed on Dec. 24, 2021, the content of which is incorporated herein in its entirety by reference.

### BACKGROUND OF THE INVENTION

### 1. Field of the Invention

The present invention relates to a method and a platform for disrupting intracellular microtubules.

### 2. The Prior Art

Microtubules (MTs) are the main cytoskeleton, which regulates various cellular physiological functions in various cells. In order to study the function of microtubules, the traditional method is to add microtubule binding drugs such as nocodazole and colchicine to disrupt the microtubules, and then the effect on cells is explored. Because these drugs can disrupt microtubules and inhibit cell growth, they have been used extensively in chemotherapeutics. However, these drugs disrupt the microtubules very slowly (it requires 106.86±12.11 minutes for nocodazole (3.3 µM) to disrupt half of the intracellular microtubules, and it requires 94.28±8.63 minutes for colchicine (500 µM) to disrupt half of the intracellular microtubules), and cannot specifically disrupt the microtubule structures in a specific region, such as primary cilia, centrosome, mitotic spindle and intercellular bridge. Therefore, the usage and efficacy are limited.

In order to solve the above-mentioned problems, those skilled in the art urgently need to develop a novel and quick method for specifically disrupting intracellular microtubules for the benefit of a large group of people in need thereof.

### SUMMARY OF THE INVENTION

A primary objective of the present invention is to provide a method for disrupting intracellular microtubules, comprising the following steps: (a) expressing an artificially engineered microtubule-cleaving enzyme on a cytosol, and expressing a microtubule-binding protein on the intracellular microtubules; and (b) driving the artificially engineered microtubule-cleaving enzyme to the intracellular microtubules to dimerize with the microtubule-binding protein by using a chemical component, thereby allowing the artificially engineered microtubule-cleaving enzyme to recruit onto the intracellular microtubules and specifically disrupt a structure of the intracellular microtubules.

According to an embodiment of the present invention, the chemical component is a macrolide compound or a tetracyclic diterpene compound.

According to an embodiment of the present invention, the macrolide compound is rapamycin.

According to an embodiment of the present invention, when the chemical component is rapamycin, the artificially engineered microtubule-cleaving enzyme comprises FK506-binding protein (FKBP).

According to an embodiment of the present invention, when the chemical component is rapamycin, the microtubule-binding protein is FKBP-rapamycin binding domain (FRB).

According to an embodiment of the present invention, the tetracyclic diterpene compound is gibberellin.

According to an embodiment of the present invention, when the chemical component is gibberellin, the microtubule-binding protein is Gibberellin insensitive protein (GAIs).

According to an embodiment of the present invention, when the chemical component is gibberellin, the artificially engineered microtubule-cleaving enzyme comprises mammalian optimized Gibberellin insensitive dwarf1 (mGID1).

According to an embodiment of the present invention, the artificially engineered microtubule-cleaving enzyme further comprises an artificially engineered spastin.

According to an embodiment of the present invention, three amino acid residues of the artificially engineered spastin are mutated into three consecutive glutamines by site-directed mutagenesis, and the artificially engineered spastin is a truncated spastin lacking 1st to 140th amino acids at N-terminus.

According to an embodiment of the present invention, the structure of the intracellular microtubules is primary cilia, mitotic spindle or intercellular bridge.

According to an embodiment of the present invention, the primary cilia comprises an axoneme and a ciliary membrane, and when the primary cilia is disrupted, the ciliary membrane is in a bulging and branched phenotype.

According to an embodiment of the present invention, the intracellular microtubules are disrupted within one hour.

According to an embodiment of the present invention, the intracellular microtubules are disrupted in a reversible manner.

According to an embodiment of the present invention, each of the intracellular microtubules is a tyrosinated microtubule.

According to an embodiment of the present invention, the FRB tags an A1AY1 protein.

Another objective of the present invention is to provide a method for disrupting intracellular microtubules, comprising the following steps: (a) expressing an artificially engineered microtubule-cleaving enzyme on a cytosol, and expressing a plurality of microtubule-binding proteins on the intracellular microtubules; and (b) using a light to stimulate the artificially engineered microtubule-cleaving enzyme and the plurality of microtubule-binding proteins, wherein the light induces dimerization of the plurality of microtubule-binding proteins and the artificially engineered microtubule-cleaving enzyme, thereby allowing the artificially engineered microtubule-cleaving enzyme to recruit onto the intracellular microtubules and specifically disrupt a structure of the intracellular microtubules.

According to an embodiment of the present invention, the light is blue light.

According to an embodiment of the present invention, the plurality of microtubule-binding proteins are cryptochrome 2 (Cry2) and N-terminal 170 amino acids of calcium and integrin-binding protein 1 (C1B1)(CIBN).

According to an embodiment of the present invention, the artificially engineered microtubule-cleaving enzyme comprises an artificially engineered spastin.

According to an embodiment of the present invention, the structure of the intracellular microtubules is primary cilia, mitotic spindle or intercellular bridge.

According to an embodiment of the present invention, the primary cilia comprises an axoneme and a ciliary membrane, and when the primary cilia is disrupted, the ciliary membrane is in a bulging and branched phenotype.

According to an embodiment of the present invention, the intracellular microtubules are disrupted within one hour.

According to an embodiment of the present invention, the artificially engineered microtubule-cleaving enzyme specifically disrupts the structure of the intracellular microtubules in an illuminated region.

According to an embodiment of the present invention, the intracellular microtubules are disrupted in a reversible manner.

Another objective of the present invention is to provide a platform for disrupting intracellular microtubules, being established by the aforementioned method.

In summary, the method and the platform for disrupting intracellular microtubules have the following effect. They can accurately and quickly disrupt the microtubule structures in specific areas of cells by adding specific chemical components or specific wavelengths of light. In addition to providing important reagents for microtubule-related research in basic science, it may even be developed into a new technology for precise chemotherapy. Compared with two common traditional microtubule-binding drugs, nocodazole and colchicine, the present invention can disrupt intracellular microtubules faster (8.53 to 9.67 times faster), and remove more intracellular microtubules within one hour (3.3 µM nocodazole removes 31% of microtubules; 500 µM colchicine removes 49% of microtubules; the present invention removes 93% of microtubules). Also, the present invention can precisely disrupt specific microtubule structures, such as primary cilia, mitotic spindle and intercellular bridge, and solve the technical limitation that traditional microtubule binding drugs cannot specifically disrupt microtubule structures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included here to further demonstrate some aspects of the present invention, which can be better understood by reference to one or more of these drawings, in combination with the detailed description of the embodiments presented herein.

FIG. 1A shows that recruitment of the artificially engineered microtubule-cleaving enzyme (i.e., artificially engineered spastin) onto microtubules (MTs) leads to their rapid disassembly, illustrating the inducible microtubule (MT) disassembly system; PM represents plasma membrane; MT represents microtubule; MAP represents MT-associated protein; dimerizing domain comprises three combinations: FK506-binding protein (FKBP) and FKBP-rapamycin binding domain (FRB), Gibberellin insensitive protein (GAIs) and mammalian optimized Gibberellin insensitive dwarf1 (mGID1), and cryptochrome 2 (Cry2) and N-terminal 170 amino acids of calcium and integrin-binding protein 1 (C1B1)(CIBN).

FIG. 1B shows the MT-binding domain of ensconsin-cyan fluorescent protein-FKBP-rapamycin binding domain (EMTB—CFP—FRB) localizes to cytosolic MTs.

FIG. 1C shows the normalized intensity profiles of α-tubulin (solid line) and EMTB—CFP—FRB (dotted line) along the dotted lines drawn in FIG. 1B.

FIG. 1D shows addition of rapamycin (Rapa) rapidly translocated yellow fluorescent protein-tagged FKBP (YFP—FKBP) onto EMTB—CFP—FRB-labeled MTs and increased the fluorescence resonance energy transfer (FRET) signal, in which HeLa cells co-transfected with EMTB—CFP—FRB and YFP—FKBP were treated with 100 nM rapamycin (Rapa); scale bar, 10 µm; the color bar on the right indicates the brightness value represented by the color of the FRET signal.

FIG. 1E shows the normalized intensity of FRET/CFP in cells before and after rapamycin and 0.1% dimethyl sulfoxide (DMSO)(control) treatment; n = 6 and 10 cells in the rapamycin and DMSO group, respectively, from three independent experiments; the solid line represents DMSO, and the dotted line represents Rapa.

FIG. 2A shows HeLa cells transfected with YFP-tagged full-length spastin (SpastinFL-YFP; green) and truncated spastin (dNSpastin-YFP; green; the amino acid sequence of dNSpastin is SEQ ID NO:2) were fixed and stained with the α-tubulin antibody (red); YFP alone and an ED (enzyme dead) form of each enzyme (SpastinFLED (SEQ ID NO:3) and dNSpastinED (SEQ ID NO:4)) are negative controls; scale bar, 10 µm.

FIG. 2B shows HeLa cells co-transfected with EMTB-CFP-FRB and the indicated constructs were treated with either 0.1% DMSO or 100 nM rapamycin (Rapa) for 1 hr and then were fixed and stained with anti-α-tubulin antibody; scale bar, 10 µm; dotted lines highlight transfected cells; removal of the MT-binding domain from dNSpastin (while retaining its catalytic domain; dNSpastinCD-YFP-FKBP; the amino acid sequence of dNSpastinCD is SEQ ID NO:5); mutation of key residues that are required for MT binding of Spastin (dNSpastin3Q-YFP-FKBP; the amino acid sequence of dNSpastin3Q is SEQ ID NO:6); and tagging spastin with a plasma membrane-binding sequence, C2 domain of Lactadherin (C2Lact).

FIG. 2C shows the normalized intensity of α-tubulin in cells expressing the indicated construct with 0.1% DMSO or rapamycin treatment; n = 103, 39, 33, 123, 41, 175, 144, 88, 79, 82, 90, 134, 104, 81, and 97 cells from left to right; three to five independent experiments; data are shown as the mean ± S.E.M; Student’s t-tests were performed, with p-values indicated.

FIG. 2D shows rapidly recruiting engineered spastins onto MTs, in which HeLa cells co-transfected with EMTB—CFP—FRB (blue) and either dNSpastin3Q-YFP-FKBP or dNSpastin-YFP-FKBP-C2Lact (green) were treated with 100 nM rapamycin (Rapa); the FRET signal of cells was monitored in real time by live-cell imaging; scale bar, 10 µm.

FIG. 2E shows the normalized intensity of FRET/CFP in cells upon rapamycin treatment; n = 23 and 25 cells for dNSpastin3Q-YFP-FKBP and dNSpastin-YFP-FKBP-C2Lact, respectively.

FIG. 2F shows the video frames of MTs after recruitment of the artificially engineered microtubule-cleaving enzyme (i.e., spastin) onto MTs, leading to their rapid disassembly; HeLa cells co-transfected with the indicated constructs were treated with rapamycin (100 nM); scale bar, 10 µm.

FIG. 2G shows recruitment of the artificially engineered microtubule-cleaving enzyme (i.e., artificially engineered spastin) onto MTs leads to their rapid disassembly, in which the normalized MT filament area in cells with different MT disruption treatments; n = 30, 45, 19, 20, 14, and 6 cells in nocodazole (3.3 µM), colchicine (500 µM), dNSpastin-C2Lact, dNSpastin3Q, dNSpastin3QED, and dNSpastin3Q with MG132 (an effective, reversible and cell-permeable proteasome inhibitor) (50 µM) pre-treatment, respectively, from three independent experiments; data are shown as the mean ± S.E.M.

FIG. 2H shows the MT disruption rates of MT-targeting agents (MTAs) and the MT disruption system of the present invention, in which HeLa cells transfected with the indicated constructs were treated with nocodazole (Noc)(3.3 µM, a type of MTA), colchicine (Col)(500 µM, a type of MTA), or rapamycin (Rapa)(100 nM); scale bar, 10 µm.

FIG. 2I shows the MT disruption rates of MTAs and the MT disruption system of the present invention, in which the half time of MT disassembly triggered by the indicated MT disruption systems; n = 30, 45, 19, 30, and 6 cells from left to right, three to five independent experiments.

FIG. 2J shows the MT disruption rates of MTAs and the MT disruption system of the present invention, in which the relative proportion of MT area left in transfected HeLa cells after 1 hr of different MT-disrupting treatments. n = 34, 30, 45, 19, 20, 6, and 14 cells from left to right, three to five independent experiments.

FIG. 2K shows rapid disruption of MTs in COS7 cells, in which COS7 cells transfected with dNSpastin3Q-YFP-FKBP (green) and EMTB—CFP—FRB (blue) were treated with rapamycin (100 nM) and imaged; scale bar, 10 µm.

FIG. 2L shows rapid disruption of MTs in COS7 cells, in which the normalized MT filament area in cells transfected and treated as in FIG. 2K; n = 12 cells.

FIG. 2M shows rapid disruption of MTs in U2OS cells, in which U2OS cells transfected with dNSpastin3Q-YFP-FKBP (green) and EMTB-CFP-FRB (blue) were treated with rapamycin (100 nM) and imaged; scale bar, 10 µm.

FIG. 2N shows rapid disruption of MTs in U2OS cells, in which the normalized MT filament area in cells transfected and treated as in FIG. 2M; n = 16 cells.

FIG. 2O shows using a gibberellin-based system to rapidly translocate proteins of interest onto MTs, in which HeLa cells co-transfected with EMTB-CFP-mammalian optimized Gibberellin insensitive dwarf1 (mGID1)(blue) and YFP-Gibberellin insensitive protein (GAIs) (green) (mGID1 and GAIs are dimerizing partners of the gibberellin system) were treated with GA3-AM (chemical dimerizer of the gibberellin system; 100 µM); live-cell imaging was used to monitor the FRET/CFP signal in cells; scale bar, 10 µm.

FIG. 2P shows using a gibberellin-based system to rapidly translocate proteins of interest onto MTs, in which the normalized ratio of FRET/CFP in cells upon GA3-AM (blue) and 0.1% DMSO (red) treatment; n = 10 and 6 cells for the DMSO and GA3-AM treatments, respectively.

FIG. 2Q shows using the gibberellin-based system to rapidly disassemble MTs, in which HeLa cells co-transfected with dNSpastin3Q-YFP-GAIs (green) and EMTB-CFP-mGID1 (black and blue) were treated with GA3-AM (100 µM); scale bar, 10 µm.

FIG. 2R shows using the gibberellin-based system to rapidly disassemble MTs, in which the normalized MT filament area in cells transfected with the constructs shown in FIG. 2Q upon GA3-AM treatment; n = 8 cells.

FIG. 2S shows inhibition of spastin activity reverses MT disruption, in which HeLa cells co-transfected with EMTB—CFP—FRB (black) and dNSpastin3Q-YFP-FKBP (green) were pre-treated with rapamycin for 28 min to induce acute MT disassembly; spastazoline (10 µM), a spastin inhibitor, was then added to the cultures to halt the MT disruption system; arrows indicate the centrosome-derived MTs; scale bar, 10 µm.

FIG. 2T shows inhibition of spastin activity reverses MT disruption, in which normalized MT filament area for cells co-transfected and treated as in FIG. 2S; n = 3 cells from two independent experiments.

FIG. 2U shows inhibition of spastin activity reverses MT disruption, in which the polymerization rates of MTs regrowing from spastin-digested MT fragments in cytosol (acentrosome) and centrosomes; n = 147 and 74 MTs in the cytosol and centrosome group, respectively.

FIG. 3A shows acute MT disassembly attenuates vesicular trafficking, in which HeLa cells co-transfected with EMTB—CFP—FRB (blue), dNSpastin3Q-mCherry-FKBP (dNSpastin3Q-mCh—FKBP) (red), and trans-Golgi network integral membrane protein 38-YFP (TGN38-YFP) (green) were treated with rapamycin (100 nM) to induce MT disruption; dotted lines indicate the cell boundary; scale bar, 10 µm.

FIG. 3B shows acute MT disassembly attenuates vesicular trafficking, in which the MT filament area in the cell shown in FIG. 3A.

FIG. 3C shows acute MT disassembly attenuates vesicular trafficking, in which trajectories of each TGN38-YFP-labeled vesicle at different levels of MT disruption; insets show higher-magnification images of the regions indicated by the dotted-line boxes; scale bar, 10 µm.

FIG. 3D shows acute MT disassembly attenuates vesicular trafficking, in which the displacement (left) and velocity (right) of each labeled vesicle shown in FIG. 3C; n = 312, 319, and 111 vesicles in the MT 100, 50, and 0% group, respectively.

FIG. 3E shows acute MT disassembly attenuates lysosome dynamics, in which HeLa cells co-transfected with EMTB—CFP—FRB (blue), dNSpastin3Q-mCh—FKBP (red), and LAMP3-YFP (green) were treated with rapamycin (100 nM) to induce MT disruption; dotted lines indicate the cell boundary; scale bar, 10 µm.

FIG. 3F shows acute MT disassembly attenuates lysosome dynamics, in which the MT filament area in the cell shown in FIG. 3E.

FIG. 3G shows acute MT disassembly attenuates lysosome dynamics, in which trajectories of each LAMP3-YFP-labeled lysosome at different levels of MT disruption; insets show higher-magnification images of the regions indicated by the dotted-line boxes; scale bar, 10 µm.

FIG. 3H shows acute MT disassembly attenuates lysosome dynamics, in which displacement (left) and velocity (right) of each labeled lysosome shown in FIG. 3G; n = 2893, 2618, and 2315 lysosomes in the MT 100, 50, and 0% group, respectively.

FIG. 4A shows disruption of tyrosinated MTs, in which COS7 cells transfected with TagRFP—FRB—A1AY1 (red) were labeled by anti-α-tubulin antibody (green; upper panel), anti-tyrosinated tubulin antibody (green; middle panel), or anti-detyrosinated tubulin antibody (green; lower panel), respectively.

FIG. 4B shows disruption of tyrosinated MTs, in which the intensity profiles of TagRFP—FRB—A1AY1 (red) and tubulin with the indicated PTMs (green) along dotted lines drawn in FIG. 4A; the solid line represents TagRFP—FPB—A1AY1; the dotted lines from top to bottom indicate α-tubulin, tyrosinated tubulin, and detyrosinated tubulin, respectively.

FIG. 4C shows disruption of tyrosinated MTs, in which COS7 cells co-transfected with TagRFP—FRB—A1AY1 (red) and dNSpastin3Q-TagCFP-FKBP (cyan) were treated with 0.1% DMSO or rapamycin (100 nM) for 1 hr and following by immunostaining with anti-tyrosinated tubulin antibody (green); dotted lines highlight the transfected cells; scale bar, 20 µm.

FIG. 4D shows disruption of tyrosinated MTs, in which the normalized intensity of tyrosinated MTs in TagRFP—FRB—A1AY1 and dNSpastin3Q—TagCFP—FKBP co-transfected cells upon 0.1% DMSO or rapamycin treatment for 1 hr; n=31 and 26 cells in DMSO and rapamycin treated groups, respectively.

FIG. 5A shows subcellular distribution of CFP—FRB—MAP4m at different phases of the cell cycle, in which NIH3T3 cells transfected with CFP—FRB—MAP4m (green) were serum starved to promote ciliogenesis; ciliated cells were fixed and labeled with GT335, an antibody that is specific for glutamylated tubulin (Glu-tub; red) at ciliary axonemes.

FIG. 5B shows subcellular distribution of CFP—FRB—MAP4m at different phases of the cell cycle, in which HeLa cells expressing CFP—FRB—MAP4m (green) were synchronized during metaphase (FIG. 5B) and telophase (FIG. 5C) and then were fixed and stained with α-tubulin antibody (red); dotted lines indicate cell boundaries; scale bar, 10 µm.

FIG. 5C shows subcellular distribution of CFP—FRB—MAP4m at different phases of the cell cycle, in which HeLa cells expressing CFP—FRB—MAP4m (green) were synchronized during metaphase (FIG. 5B) and telophase (FIG. 5C) and then were fixed and stained with α-tubulin antibody (red); dotted lines indicate cell boundaries; scale bar, 10 µm.

FIG. 5D shows rapid disruption of primary cilia, mitotic spindles, and intercellular bridges, in which NIH3T3 fibroblasts co-transfected with 5HT6-mCherry (5HT6-mCh; a ciliary membrane marker; red), CFP—FRB—MAP4m (blue), and dNSpastin3Q-YFP-FKBP (green) were serum starved for 24 hr to induce ciliogenesis; ciliated cells were then treated with rapamycin (100 nM) to induce dNSpastin3Q-YFP-FKBP recruitment to axonemal MTs; scale bar, 5 µm.

FIG. 5E shows rapid disruption of primary cilia, mitotic spindles, and intercellular bridges, in which the normalized length of axonemes (Axoneme), primary cilia (Cilia membrane), and dNSpastin3Q-YFP-FKBP in cilia (dNSpastin3Q-YFP-FKBP) upon rapamycin treatment; n = 6 cells.

FIG. 5F shows rapid disassembly of primary cilia, in which NIH3T3 cells co-transfected with 5HT6-mCh (a ciliary membrane marker; red), CFP—FRB—MAP4m (an axoneme marker; blue), and dNSpastin3Q-YFP-FKBP (upper panel; green) or enzyme dead dNSpastin3QED-YFP-FKBP (lower panel; green) were serum starved for 24 hr to induce ciliogenesis; ciliated cells were treated with rapamycin (100 nM) to recruit dNSpastin proteins onto axonemes; the recruitment of spastin proteins, morphology of ciliary axonemes, and ciliary membrane, upon rapamycin treatment was monitored by live-cell imaging; dotted lines indicate the cell boundary; scale bar, 5 µm.

FIG. 5G shows that the structure of the cilia occasionally does not collapse after axoneme disruption, in which NIH3T3 cells co-transfected with 5HT6-mCh (ciliary membrane; red), CFP—FRB—MAP4m (axonemal MTs; blue), and dNSpastin3Q-YFP-FKBP (green) were serum starved to induce ciliogenesis; ciliated cells were treated with rapamycin (100 nM) to recruit dNSpastin3Q-YFP-FKBP onto axonemes; the recruitment of spastin proteins, morphology of the axonemes, and ciliary membrane were monitored upon rapamycin addition by live-cell imaging; scale bar, 5 µm.

FIG. 5H shows the length of the indicated structures in the cell shown in FIG. 5G was measured and plotted; the time period during which the ciliary membrane became bulged and/or branched is shown with the gray lines above the graph.

FIG. 5I shows cells can spontaneously form ciliary membranes without axonemes, in which NIH3T3 cells were serum starved to promote ciliogenesis; ciliated cells were fixed and labeled with antibodies against Arl13b (a ciliary membrane marker; red) and against acetylated tubulin (acetylated tub; a ciliary axoneme marker; green); dotted lines indicate cell boundaries; insets show higher-magnification images of the regions indicated by the dotted-line boxes; scale bar, 10 µm.

FIG. 5J shows cells can spontaneously form ciliary membranes without axonemes, in which the percentage of cells that spontaneously formed ciliary membranes with a short or absent axoneme or a normal axoneme; n = 391 cells from four dishes.

FIG. 5K shows rapid disruption of mitotic spindles, in which HeLa cells co-transfected with H2B-mCherry (H2B-mCh; a chromosome marker; red), CFP—FRB—MAP4m (a marker of mitotic spindles; blue), and dNSpastin3Q-YFP-FKBP (green) were synchronized in metaphase and treated with rapamycin (100 nM) to induce dNSpastin3Q-YFP-FKBP recruitment to mitotic spindles; scale bar, 10 µm.

FIG. 5L shows rapid disruption of intercellular bridges, in which HeLa cells co-transfected with H2B-mCherry (red), CFP—FRB—MAP4m (intercellular bridges; blue), and dNSpastin3Q-YFP-FKBP (green) were treated with rapamycin (100 nM) to induce dNSpastin3Q-YFP-FKBP recruitment to intercellular bridges; arrows indicate the intercellular bridges; scale bar, 10 µm.

FIG. 5M shows rapid disruption of mitotic spindles, in which the normalized area of the mitotic spindle and intensity of dNSpastin3Q-YFP-FKBP in the mitotic spindle upon rapamycin treatment. n = 6 cells.

FIG. 5N shows rapid disruption of intercellular bridges, in which the normalized area of intercellular bridges and intensity of dNSpastin3Q-YFP-FKBP at intercellular bridges upon rapamycin treatment. n = 5 cells.

FIG. 5O shows spastin does not disrupt centrosomes, in which HeLa cells co-transfected with EMTB-CFP-FRB (blue), dNSpastin3Q-YFP-FKBP (green), and PACT-mCh (a centrosomal marker; red) were treated with rapamycin (100 nM) and imaged; dotted lines indicate the cell boundary; insets show higher-magnification images of the centrosome regions; scale bar, 10 µm.

FIG. 5P shows spastin does not disrupt centrosomes, in which the normalized area of MT filaments in the cytosol (Cytosol MTs) and centrosomes is shown; n = 8 cells.

FIG. 6A shows using light to disassemble MTs in a reversible and location-specific manner, in which COS7 cells co-transfected with EMTB—YFP—CIBN and mCh—Cry2 (red) were incubated with SPY650-tubulin (SPY650-tub) to visualize MTs; the cells were illuminated by blue light within a specified region (indicated by the dotted circle) for the indicated time period; scale bar, 10 µm.

FIG. 6B shows using light to disassemble MTs in a reversible and location-specific manner, in which the normalized intensity of mCh—Cry2 at MTs in illuminated regions (Light region) and non-illuminated regions (Dark region); n = 6 cells.

FIG. 6C shows using light to disassemble MTs in a reversible and location-specific manner, in which COS7 cells co-transfected with EMTB—YFP—CIBN and dNSpastin3Q-mCh—Cry2 were incubated with SPY650-tubulin to visualize MTs; the cells were illuminated by blue light as in FIG. 6A; scale bar, 10 µm.

FIG. 6D shows using light to disassemble MTs in a reversible and location-specific manner, in which the normalized intensity of dNSpastin3Q-mCh—Cry2 and mCh—Cry2 at MTs and the normalized area of SPY650-tubulin in illuminated regions and non-illuminated regions; n = 6 and 6 cells in dNSpastin3Q-mCh—Cry2 and mCh—Cry2 group, respectively.

FIG. 6E shows recruitment of light-sensitive dimerizing proteins without spastin does not disrupt MTs, in which COS7 cells were co-transfected with EMTB-YFP-CIBN and mCh—Cry2 (red), and MTs were labeled with SPY650-tubulin (SPY650-tub; black); illumination with blue light for the indicated period of time triggered rapid recruitment of mCh—Cry2 onto MTs in the light-illuminated region (dotted circle); the MTs, however, remained intact after recruitment of mCh—Cry2; scale bar, 10 µm.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

The inventors first determined whether variances were equal or not with the F-test and then used the unpaired two-tailed Student’s t-test to calculate p-values via PRISM 6 software. A p-value of <0.05 indicated a significant difference, and p < 0.01 indicated a highly significant difference.

As used herein, the term “artificially engineered microtubule-cleaving enzyme” comprises an artificially engineered spastin and a protein selected from FK506-binding protein (FKBP) or mammalian optimized Gibberellin insensitive dwarf1 (mGID1).

As used herein, the term “transformation” and the term “transfection” can be used interchangeably, and broadly refers to the manner in which a nucleic acid molecule is introduced into a selected host cell. According to techniques known in the art, a nucleic acid molecule (for example, a recombinant DNA construct or a recombinant vector) can be introduced into a selected host cell by a variety of techniques, such as calcium phosphate or calcium chloride-mediated transfection, electroporation, microinjection, particle bombardment, liposome-mediated transfection, transfection using bacteriophage or other methods.

As used herein, the terms “DNA construct”, “nucleic acid construct” and “indicated construct” can be used interchangeably, and refer to DNA molecules capable of genomic integration, comprising one or more transgene DNA sequences, which have been linked in a functional manner using well-known recombinant DNA technology.

As used herein, the term “dimerizing domain” comprises three combinations: FK506-binding protein (FKBP) and FKBP-rapamycin binding domain (FRB), Gibberellin insensitive protein (GAIs) and mammalian optimized Gibberellin insensitive dwarf1 (mGID1), and cryptochrome 2 (Cry2) and N-terminal 170 amino acids of calcium and integrin-binding protein 1 (C1B1)(CIBN).

The procedures of cell culture and transfection are as follows. African green monkey kidney cell line COS7 (CRL-1651), cervical cancer cell line HeLa (CCL-2), human osteosarcoma cell line U2OS (HTB-96), human embryonic kidney cell line HEK293T (CRL-3216), and mouse embryonic fibroblasts NIH3T3 cells (CRL-1658) were maintained at 37° C., 5% CO₂, and 95% humidity in Dulbecco’s modified Eagle’s medium (DMEM) supplemented with 10% fetal bovine serum (FBS), and penicillin and streptomycin (Corning). To induce ciliogenesis, NIH3T3 cells were serum starved for 24 hr. COS7 cells were transfected with plasmid DNAs by using TurboFect transfection reagents (Thermo Fisher). HeLa, U2OS, HEK293T, and NIH3T3 cells were transfected with FuGENE HD (Promega). Transfected cells were incubated for 24-48 hr prior to imaging and other experiments.

The procedures of DNA constructs are as follows. The inventors obtained constructs encoding the most abundantly expressed isoform of spastin in cells (58 kDa; starting at position M85 in the mouse spastin sequence; SEQ ID NO:17), a truncated form of dNSpastin that is missing the N-terminal 1-140 amino acids (the shortest active truncated version of spastin), and the enzyme-inactive versions SpastinFLED and dNSpastinED, each of which was tagged with enhanced yellow fluorescent protein (EYFP) (SpastinFL-YFP, dNSpastin-YFP, SpastinFLED-YFP, and dNSpastinED-YFP), from Dr. Carsten Janke (Institut Curie). To remove the MT binding domain of dNSpastin, the catalytic AAA domain of dNSpastin (dNSpstinCD) was amplified by PCR-based methods. Three residues of spastin were mutated to three glutamines (QQQ) (dNSpastin3Q) by site-directed mutagenesis DNA encoding individual forms of dNSpastin (dNSpastin, dNSpastinED, dNSpastinCD, and dNSpastin3Q) was then cloned into the YFP—FKBP vector (pEGFP-C1 backbone, Clontech) to generate dNSpastin—YFP—FKBP, dNSpastinCD-YFP-FKBP, and dNSpastin3Q-YFP—FKBP. Using this method, the inventors also generated the enzyme-inactive dNSpastin3QED-YFP-FKBP construct. dNSpastin—YFP—FKBP—C2 domain of Lactadherin (C2Lact) was generated by inserting the C2Lact sequence between the HindIII and BamHI restriction sites in dNSpastin—YFP—FKBP. The DNA fragments of A1AY1, TagRFP (SEQ ID NO:18), and TagCFP were synthesized with codon optimization and subcloned to FRB and FKBP vectors. The inventors transformed each construct into competent cells and isolated single clones for DNA purification. All DNA constructs were verified by DNA sequencing.

The procedures of immunofluorescence staining are as follows. Cells cultured in borosilicate glass Lab-Tek eight-well chambers (Nunc) were fixed in 4% paraformaldehyde (Electron Microscopy Sciences) at room temperature for 15 min. Fixed cells were permeabilized with 0.1% Triton X-100 and then incubated in blocking solution (phosphate-buffered saline with 2% bovine serum albumin) for 30 min at room temperature. To label cytosolic MTs, primary ciliary membrane, and axonemal MTs, cells were incubated for 1 hr at room temperature with mouse antibody against α-tubulin (1:500; Sigma Aldrich, T6199), rabbit antibody against Arl13b (1:500; Proteintech, 17711-1-AP), mouse antibody against glutamylated tubulin (1:100; Adipogen, AG-20B-0020-C100), mouse antibody against acetylated tubulin (1:500; Sigma Aldrich, T7451), rat antibody against tyrosinated tubulin (1:100; Sigma Aldrich, MAB1864), and mouse antibody against detyrosinated tubulin (1:100; MERCK, AB3201) each of which was diluted in blocking solution. Cells were then washed with PBS and incubated for 1 hr with appropriate secondary antibodies (1:1000 dilution; Thermo Fisher) at room temperature.

The procedures of Western blotting are as follows. HEK293T cells were co-transfected with EMTB-CFP-FRB and YFP-FKBP, dNSpastin3Q-YFP-FKBP, or dNSpastin3QED-YFP-FKBP. Two days after transfection, cells were incubated with 50 µM MG132 (an effective, reversible and cell-permeable proteasome inhibitor) (Sigma Aldrich) for 30 min and then treated with 100 nM rapamycin or 0.1% DMSO (vehicle control) for 30 min prior to cell collection. For the cold treatment (4° C.), untransfected HEK293T cells were put on ice for 40 min to depolymerize MTs. Cells were lysed in RIPA lysis buffer (50 mM Tris-HCl, pH 7.6; 2 mM EGTA; 9% NaCl; 1% Triton X-100) containing protease inhibitors (Roche). Protein concentrations were measured with the Bio-Rad Protein Assay. Cell lysates were diluted with 2× Laemmli sample buffer (Bio-Rad) and boiled at 95° C. for 10 min and then underwent western blotting. After the transfer process, the PVDF membranes (Bio-Rad) were incubated with blocking buffer (5% skim milk in Tris-buffered saline with Tween 20; TBST) for 1 hr at room temperature and then stained with primary antibodies against α-tubulin (1:1000; Sigma Aldrich, T6199) and GAPDH (1:5000; Cell Signaling, 2118), which were diluted with blocking buffer, overnight at 4° C. Membranes were washed with TBST and then were incubated with horseradish peroxidase-conjugated secondary antibodies diluted in blocking buffer (anti-rabbit, 1:10000; anti-mouse, 1:5000) for 1 hr at room temperature. The bioluminescence signal was detected with Amersham™ ECL Select™ (GE Healthcare), and blot images were acquired with an iBright™ FL1500 Instrument (Thermo Fisher).

The procedures of live-cell imaging are as follows. Live transfected cells cultured on poly(d-lysine)-coated glass coverslips (Hecht Assistent) were treated with either 100 nM rapamycin for rapid induction of protein dimerization and translocation or MT-targeting agents (10 µM nocodazole or 500 µM colchicine) during imaging. Live-cell imaging was conducted on a Nikon T1 inverted fluorescence microscope (Nikon) with a 60× oil objective (Nikon), a Prime camera (Photometrics), and 37° C., 5% CO₂ heat stage (Live Cell Instrument). Rapid recruitment of proteins of interest (POIs) was imaged at 5- or 10-sec intervals, whereas the process of MT disruption was imaged at 1-min intervals. Images were obtained using Nikon NIS-Elements AR software and processed with Huygens Deconvolution Software (Scientific Volume Imaging). Image analysis was mainly performed with Nikon NIS-Elements AR software.

The procedures of photostimulation are as follows. COS7 cells were plated on poly(d-lysine)-coated coverslips and cultured in six-well plates (Thermo Scientific) for 48 hr. Before imaging, cells were incubated with SPY650-tubulin (1000-fold dilution; Spirochrome) at 37° C. for 1 hr. Local photostimulation was carried out with a fluorescence microscope (Nikon) equipped with a digital micromirror device, polygon 400 (MIGHTEX), and a 488-nm light source. Cells were illuminated with the blue light (5 sec on/1 sec off; 1.6 nW/µm²) for the indicated duration. The mCherry and SPY650-tubulin were simultaneously imaged during photostimulation using Nikon element AR software.

The procedures of measurement of MT filament area are as follows. The MT filaments in living cells were labeled with EMTB-CFP-FRB and imaged in real time (RAW image). The rolling ball correction was used to remove the cytosol background from the RAW images, which was carried out with the Nikon NIS-Elements AR software. Images were processed to generate the binary MT filament pattern via the Otsu threshold and analyzed by Fiji software.

The procedures of tracking vesicles and lysosomes are as follows. The displacement and velocity of lysosome-associated membrane glycoprotein 3-YFP/ trans-Golgi network integral membrane protein 38-YFP (LAMP3-YFP/ TGN38-YFP) were tracked and analyzed by the DoG detector and Simple LAP tracker in the Fiji software plugin Trackmate. Estimated blob diameter was set at 0.8-1 µm, linking max distance at 2 microns, gap-closing distance at 2 µm, and gap-closing max frame gap at 0.

The procedures of cell synchronization are as follows. Plasmid DNA (i.e., DNA construct) transfection was carried out 20-24 hr prior to cell cycle synchronization. For synchronization, HeLa cells were treated with 2 mM thymidine (Sigma) for 16-18 hr to induce arrest at G1/S phase and then were treated with 2.5 ng/ml RO3306 (a selective ATP-competitive inhibitor of CDK1)(Sigma) for 12 hr to induce arrest at G2/M phase. After being washed with warm DMEM, cells were incubated with DMEM and 10% FBS at 37° C., 5% CO₂ for 30-60 min to enrich the population of metaphase and telophase cells.

### Example 1

### Rapid Translocation of Proteins of Interest (POIs) Onto Microtubules (MTs)

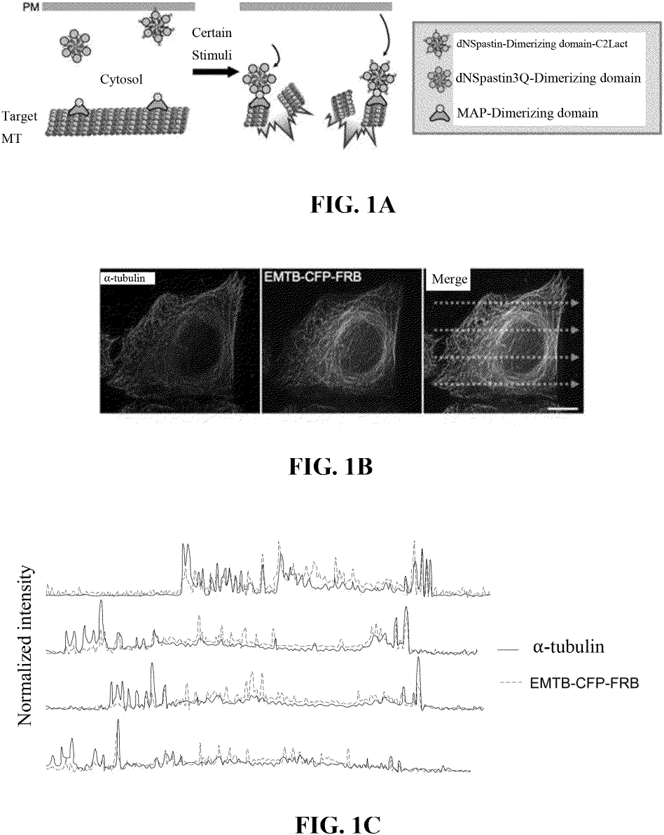

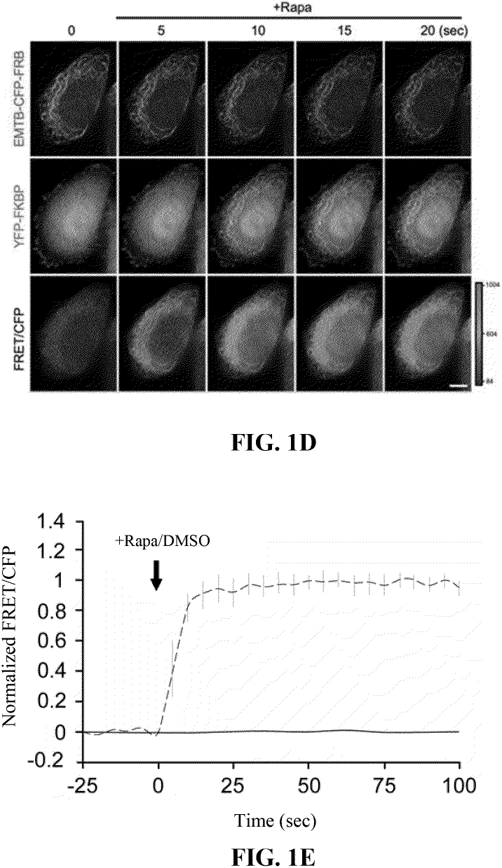

The chemically inducible dimerization (CID) system has been used to manipulate cellular signaling and molecular composition over both space and time (see R. DeRose, T. Miyamoto, T. Inoue, Manipulating signaling at will: chemically-inducible dimerization (CID) techniques resolve problems in cell biology. Pfl{ü}gers Arch. Eur. J. Physiol. 465, 409-417 (2013); S.-R. R. Hong et al., Spatiotemporal manipulation of ciliary glutamylation reveals its roles in intraciliary trafficking and Hedgehog signaling. Nat. Commun. 9, 1-13 (2018); C.-H. H. Fan et al., Manipulating Cellular Activities Using an Ultrasound-Chemical Hybrid Tool. ACS Synth. Biol. 6, 2021-2027 (2017)). This is achieved by the rapid recruitment of proteins of interest (POIs)(see dNSpastin-Dimerizing domain-C2Lact and dNSpastin3Q-Dimerizing domain in FIG. 1A) onto specific subcellular sites (see Target MT in FIG. 1A) or their substrates (i.e., MT). Dimerization of the FK506-binding protein (FKBP) and the microtubule-binding protein (i.e., FKBP-rapamycin binding domain (FRB)) that is triggered by a small chemical component, rapamycin, is one well-established CID system. To rapidly recruit POIs onto MTs, the inventors tagged FRB with a MT-binding sequence (see MAP in FIG. 1A), EMTB (the MT-binding domain of ensconsin, a type of MAP shown in FIG. 1A), and a cyan fluorescent protein (CFP) for visualizing its distribution (FIG. 1A). FIG. 1A shows that recruitment of the artificially engineered microtubule-cleaving enzyme (i.e., spastin) onto microtubules (MTs) leads to their rapid disassembly, illustrating the inducible microtubule (MT) disassembly system, in which PM represents plasma membrane. As shown in FIG. 1A, one of the dimerization proteins (i.e., FKBP or FRB) is fused with one of two engineered spastin enzymes, dNSpastin3Q and C2Lact-tagged dNSpastin, whereas the other is fused with MT-associated proteins (MAPs). Dimerization upon certain stimuli (e.g., chemical treatments or illumination) induces recruitment of engineered spastins onto MAP-labeled MTs. Acute accumulation of spastins on MTs rapidly induces disassembly of target MTs.

A linescan analysis showed that the resulting construct, EMTB—CFP—FRB, colocalizes with cytosolic MTs labeled by an antibody against α-tubulin (see FIGS. 1B and 1C). FIGS. 1B and 1C show the MT-binding domain of ensconsin-cyan fluorescent protein-FKBP-rapamycin binding domain (EMTB—CFP—FRB) localizes to cytosolic MTs. As shown in FIG. 1B, HeLa cells expressing EMTB—CFP—FRB (green) were fixed and stained with anti-α-tubulin antibody (red). Scale bar, 10 µm. As shown in FIG. 1C, the normalized intensity profiles of α-tubulin (solid line) and EMTB—CFP—FRB (dotted line) along the dotted lines drawn in FIG. 1B. Subsequently, the addition of rapamycin to HeLa cells led to the rapid translocation of yellow fluorescent protein-tagged FKBP (YFP—FKBP) onto EMTB—CFP—FRB-labeled MTs as evidenced by an increased FRET (fluorescence resonance energy transfer) signal on MTs (T_{½} of translocation: 5.73 ± 0.54 sec; FIGS. 1D and 1E). FIG. 1D shows addition of rapamycin (Rapa) rapidly translocated yellow fluorescent protein-tagged FKBP (YFP—FKBP) onto EMTB—CFP—FRB-labeled MTs and increased the fluorescence resonance energy transfer (FRET) signal, in which HeLa cells co-transfected with EMTB—CFP—FRB and YFP—FKBP were treated with 100 nM rapamycin (Rapa); scale bar, 10 µm. EMTB—CFP—FRB and YFP—FKBP were expressed in cells at the same time, so as a result of co-transfection, EMTB—CFP—FRB would continue to be located on the microtubules (filament structure) in the cells, while YFP—FKBP is freely flowed in the cytosol. Treatment of rapamycin would cause dimerization of FRB and FKBP, so YFP—FKBP can quickly accumulate on the microtubules where EMTB—CFP—FRB is located. Therefore, after treating rapamycin, YFP-FKBP would immediately become a filamentous pattern of EMTB—CFP—FRB. In addition, when the distance between CFP and YFP is close, the FRET signal would be increased. Here, the low FRET signal is represented by a cool tone, and the high signal is represented by a hot tone. The color bar indicates the signal intensity represented by various colors. FIG. 1E shows the normalized intensity of FRET/CFP in cells before and after rapamycin (Rapa) and 0.1% dimethyl sulfoxide (DMSO)(control) treatment; n = 6 and 10 cells in the rapamycin and DMSO group, respectively, from three independent experiments.

The results in this example demonstrated that cytosolic POIs can be rapidly translocated onto MTs through inducible protein dimerization. Corresponding to FIG. 1A, it means that the dNSpastin3Q-dimerizing domain protein is originally in the cytosol. Adding rapamycin can quickly accumulate the dNSpastin3Q-dimerizing domain protein on the microtubules. FIG. 1A shows two examples of dimerization. The first is the dNSpastin3Q-dimerizing domain (located in the cytosol), which can quickly accumulate to the microtubules where the MAP-dimerizing domain is located and disrupt the structure of MTs under the addition of chemical molecules and light. The second is dNSpastin-dimerizing domain-C2Lact (embedded on the cell membrane), which can quickly accumulate to the microtubules where the MAP-dimerizing domain is located and disrupt the structure of MTs under the addition of chemical molecules and light.

### Example 2

### Using Artificially Engineered Microtubule-Cleaving Enzyme for Precise MT Disruption

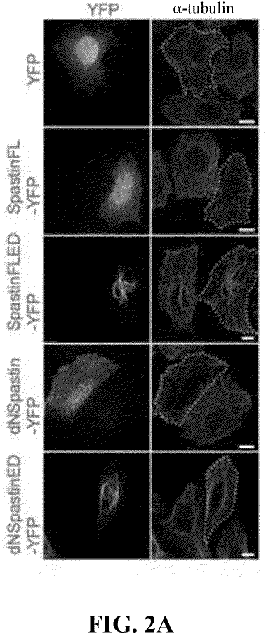

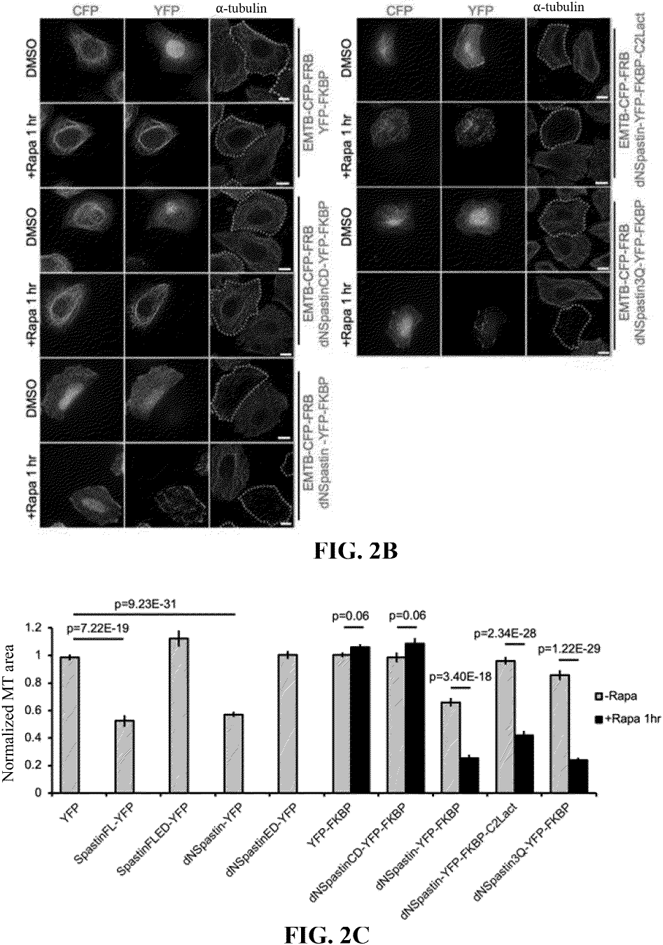

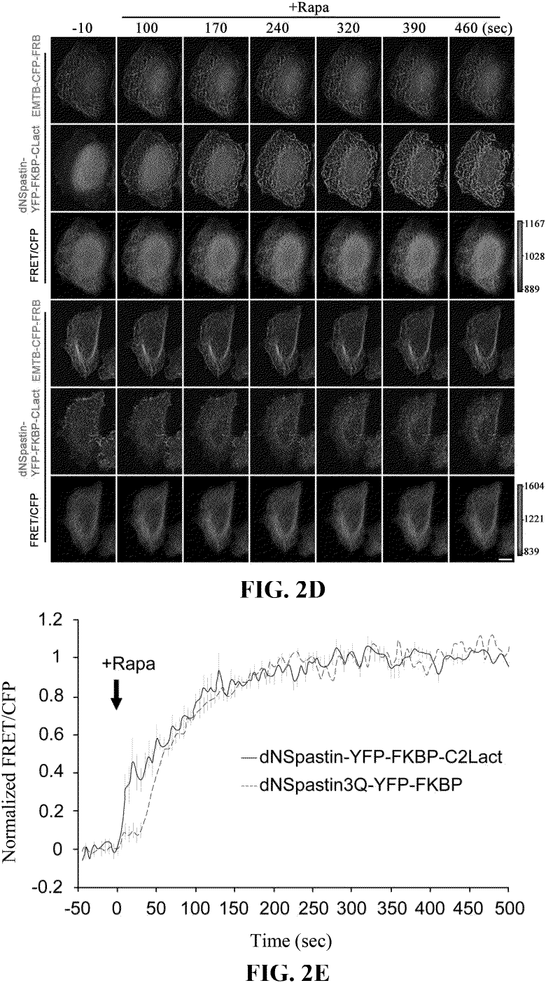

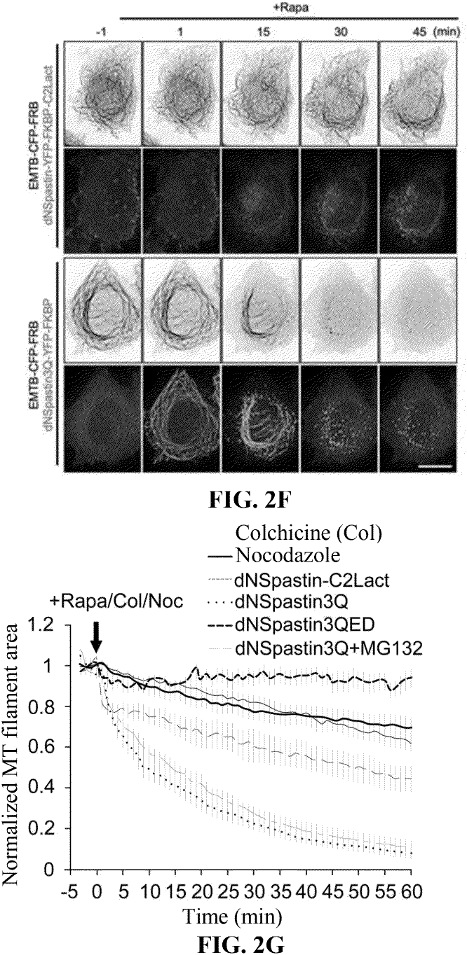

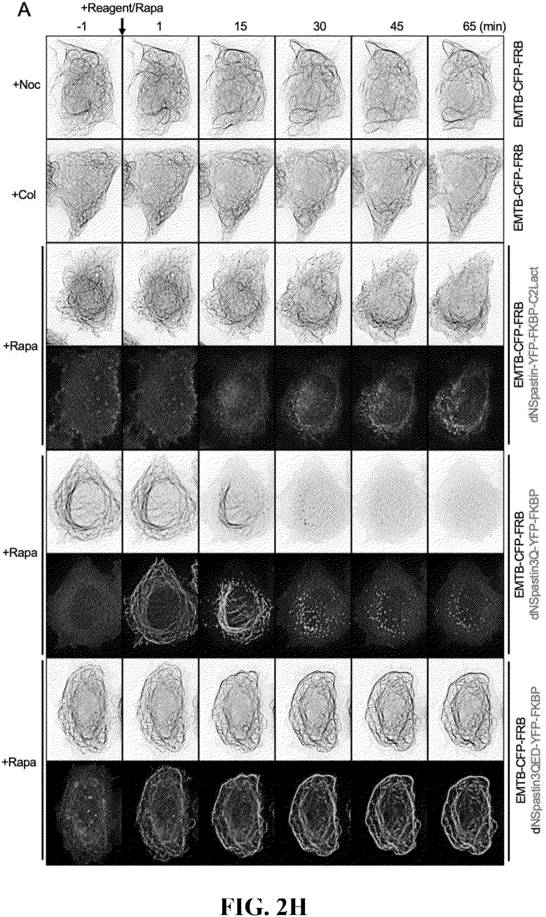

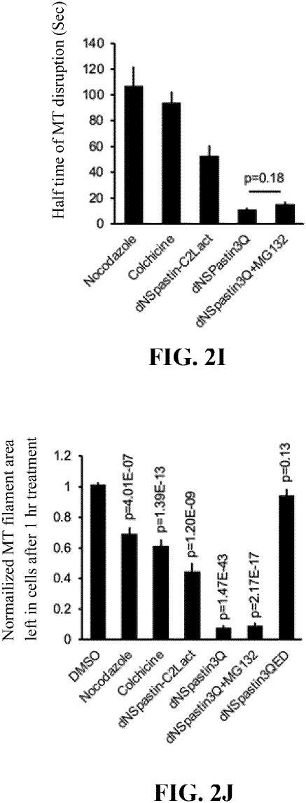

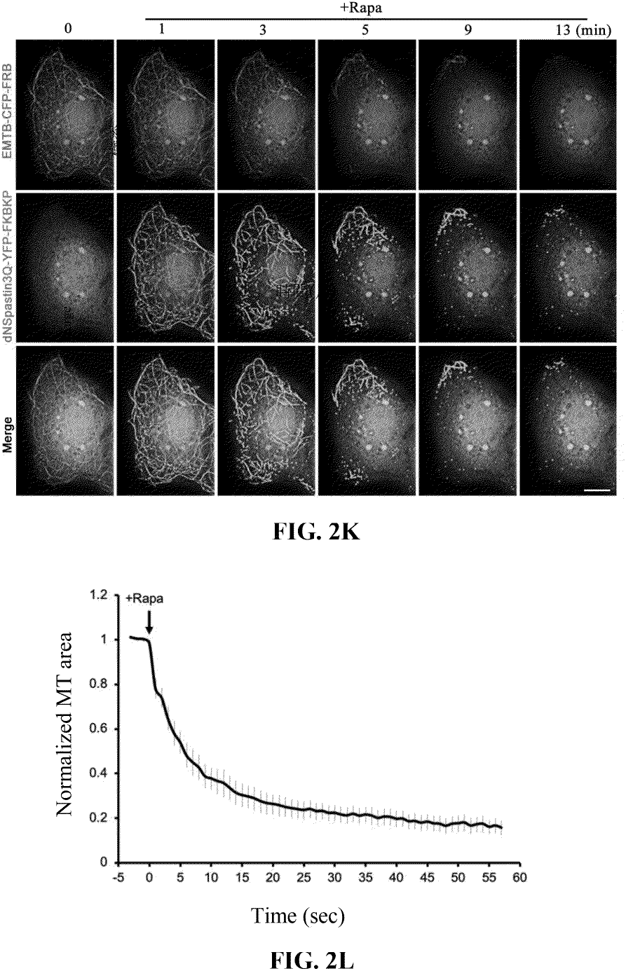

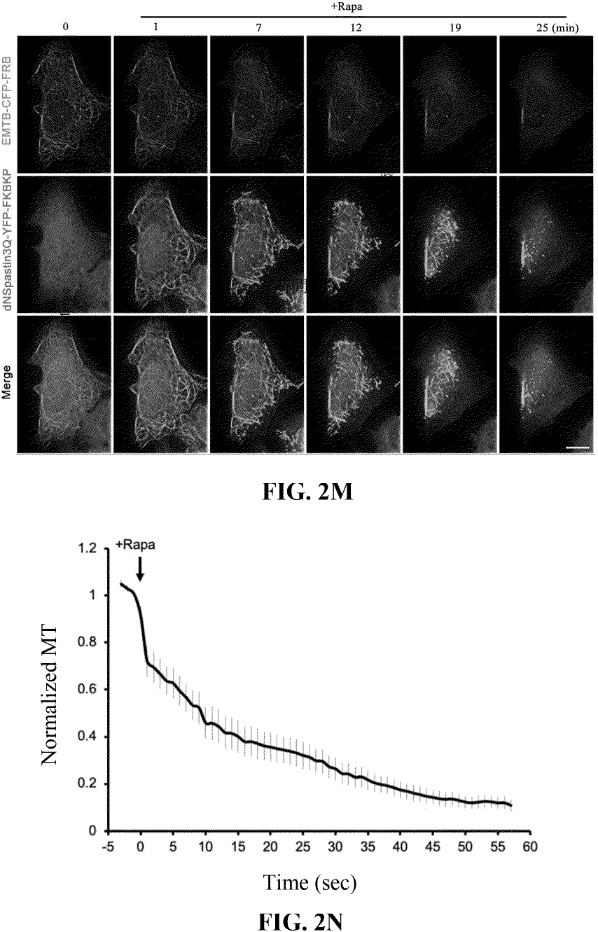

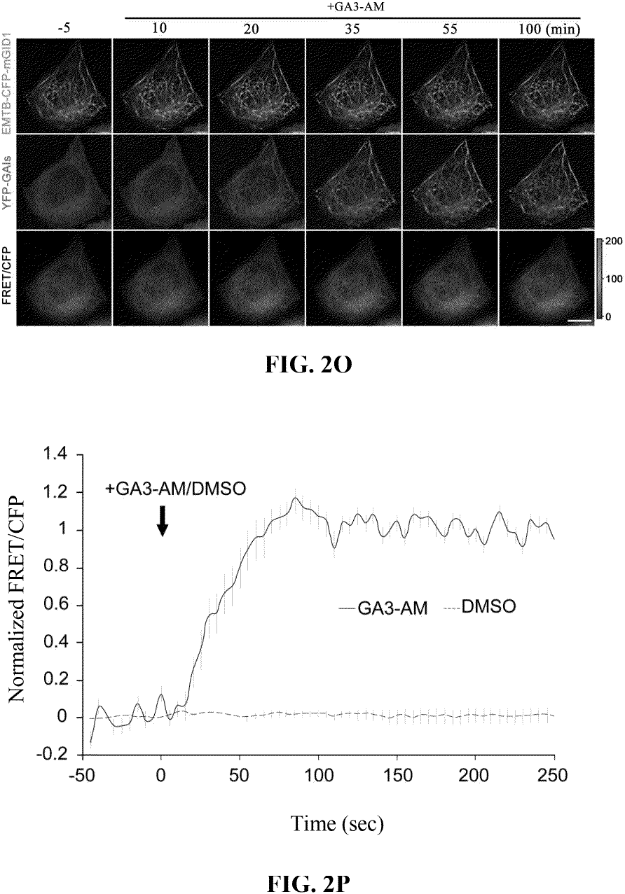

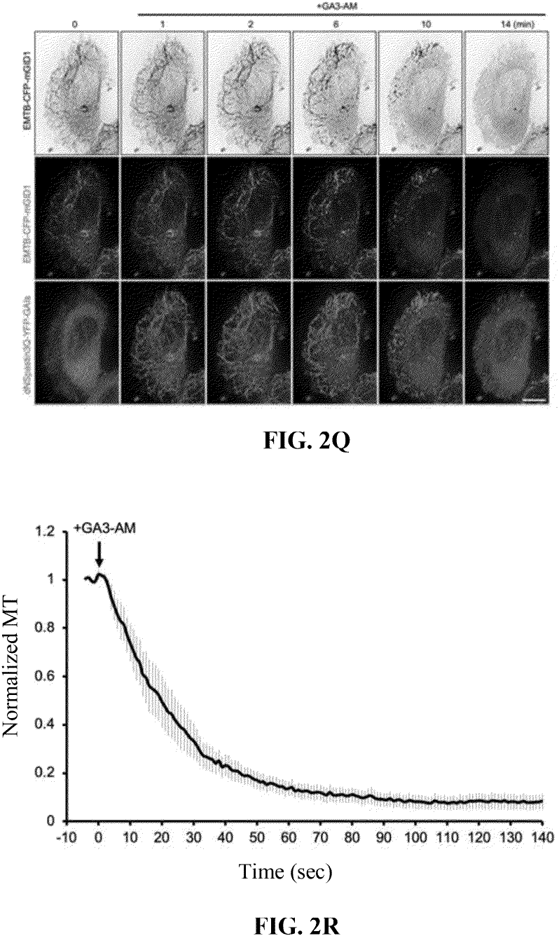

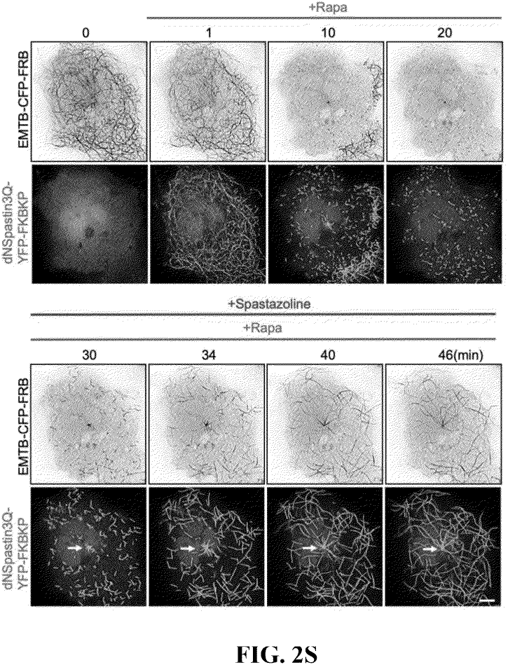

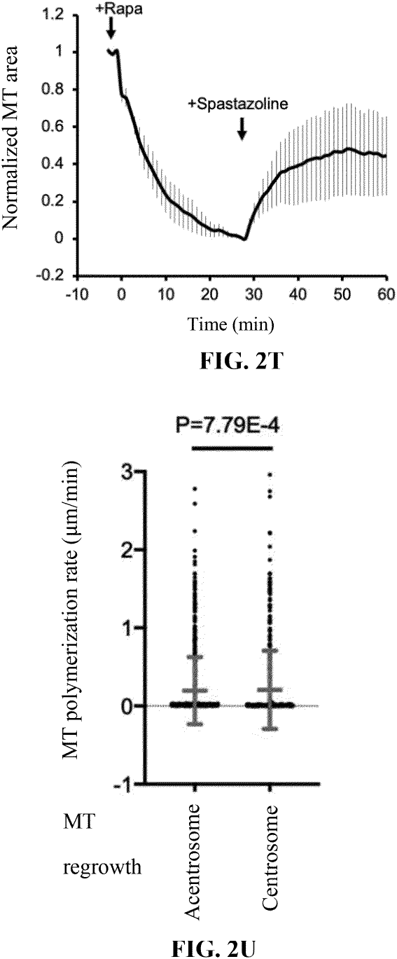

Spastin is a MT-cleaving enzyme that is ubiquitous in nearly all eukaryotic cells. Expression of full-length spastin (SpastinFL-YFP; the amino acid sequence of spastin is SEQ ID NO: 1) and truncated spastin without the N-terminal 1-140 amino acids (dNSpastin-YFP) in HeLa cells removed significant amounts of cytosolic MTs, 47.45% and 42.86%, respectively, relative to YFP-transfected control cells (See FIGS. 2A to 2C). This means that both full-length spastin and truncated spastin without N-terminal 1-140 amino acids have the effect on disrupting microtubules. FIGS. 2A to 2C show the microtubule cleavage activity of artificially engineered spastin enzyme. FIG. 2A shows HeLa cells transfected with YFP-tagged full-length spastin (SpastinFL-YFP; green) and truncated spastin (dNSpastin-YFP; green; the amino acid sequence of dNSpastin is SEQ ID NO:2) were fixed and stained with the α-tubulin antibody (red); YFP alone and an ED (enzyme dead without activity) form of each enzyme (SpastinFLED and dNSpastinED (SEQ ID NO:4)) are negative controls, and the amino acid sequence of Spastin ED (i.e., SpastinFLED) is SEQ ID NO:3; scale bar, 10 µm. The dotted line on the right indicates the periphery of the cell that expresses spastin. This figure shows whether different spastin proteins can disrupt intracellular microtubules (red). SpastinFL expressed in cells can disrupt the intracellular microtubules. Making SpastinFL be located in the cytosol, truncated spastin would also disrupt the intracellular microtubules, so it would also be located in the cytosol. This experiment shows that the full-length and truncated spastin have the same cleaving ability and can disrupt the intracellular microtubules. The truncated spastin is used in the subsequent experiments. FIG. 2B shows HeLa cells co-transfected with EMTB—CFP—FRB and the indicated constructs were treated with either 0.1% DMSO or 100 nM rapamycin (Rapa) for 1 hr and then were fixed and stained with anti-α-tubulin antibody; scale bar, 10 µm; dotted lines highlight transfected cells. As shown in FIG. 2B, adding rapamycin can indeed promote the dimerization of FRB and FKBP and make spastin disrupt microtubules. FIG. 2C shows the normalized intensity of α-tubulin in cells expressing the indicated construct with 0.1 % DMSO or rapamycin treatment; n = 103, 39, 33, 123, 41, 175, 144, 88, 79, 82, 90, 134, 104, 81, and 97 cells from left to right; three to five independent experiments; data are shown as the mean ± S.E.M; Student’s t-tests were performed, with p-values indicated. As shown in FIG. 2C, both full-length and N-terminal truncated spastin can disrupt microtubules. Compared with the YFP control group, both SpastinFL-YFP and dNSpastrin-YFP can disrupt about 40% of the microtubules. Thus the N-terminal fragment (1-140 amino acids) was dispensable for Spastin-mediated MT severing.

To minimize the MT-cleaving reaction before recruitment of spastins onto MTs, the inventors next attempted to disassociate dNSpastin from MTs by three strategies and also tagged spastin with YFP—FKBP (the amino acid sequence of FKBP is SEQ ID NO:10): 1) removal of the MT-binding domain from dNSpastin (while retaining its catalytic domain; dNSpastinCD—YFP—FKBP, wherein the amino acid sequence of dNSpastinCD is SEQ ID NO:5); 2) mutation of key residues that are required for MT binding of Spastin (dNSpastin3Q-YFP-FKBP, wherein the amino acid sequence of dNSpastin3Q is SEQ ID NO:6); and 3) tagging spastin with a plasma membrane-binding sequence, C2Lact, to mislocate spastin to the cell cortex, where MTs are rarely found (dNSpastin-YFP-FKBP-C2Lact, wherein the amino acid sequence of C2Lact is SEQ ID NO:8). The inventors then characterized the enzyme activities of the above constructs before and after they were recruited onto MTs. dNSpastin-YFP-FKBP-C2Lact and dNSpastin3Q-YFP-FKBP showed low MT-severing activities before rapamycin treatment and robustly disrupted MTs after rapamycin treatment. dNSpastinCD-YFP-FKBP did not show severing activity (see FIGS. 2A to 2C).

Live-cell imaging revealed that rapamycin treatment rapidly triggered the translocation of dNSpastin-YFP-FKBP-C2Lact (T_{½} = 55.44 ± 8.38 sec) and dNSpastin3Q-YFP-FKBP (T_{½} = 60.35 ± 5.30 sec) from the cytosol onto MTs (see FIGS. 2D and 2E). FIGS. 2D and 2E show rapidly recruiting engineered spastins onto MTs. FIG. 2D shows HeLa cells co-transfected with EMTB-CFP-FRB (blue) (the amino acid sequence of EMTB is SEQ ID NO:9, and the amino acid sequence of FRB is SEQ ID NO:11) and either dNSpastin3Q-YFP-FKBP or dNSpastin-YFP-FKBP-C2Lact (green) were treated with 100 nM rapamycin (Rapa); the fluorescence resonance energy transfer (FRET) signal of cells was monitored in real time by live-cell imaging; scale bar, 10 µm. The top three layers are cells that simultaneously express EMTB-CFP-FRB and dNSpastin3Q-YFP-FKBP. The lower three layers are cells that simultaneously express EMTB-CFP-FRB and dNSpastin-YFP-FKBP-C2Lact. The color bar on the right indicates the brightness value represented by the color of the FRET signal. FIG. 2E shows the normalized intensity of FRET/cyan fluorescent protein (CFP) in cells upon rapamycin treatment; n = 23 and 25 cells for dNSpastin3Q-YFP-FKBP and dNSpastin-YFP-FKBP-C2Lact, respectively, from four independent experiments. Data are shown as the mean ± S.E.M. It was noteworthy that MT disruption triggered by dNSpastin3Q-YFP-FKBP upon rapamycin treatment (T_{½} = 11.05 ± 1.52 min) was much faster than that of dNSpastin-YFP-FKBP-C2Lact upon rapamycin treatment (T_{½} = 53.08 ± 8.08 min), and treatment of two common MTAs, nocodazole (10 µM; T_{½} = 54.02 ± 10.29 min), colchicine (500 µM; T_{½} = 94.28 ± 8.63 min; see FIGS. 2F to 2J). FIG. 2F shows the video frames of MTs after recruitment of the artificially engineered microtubule-cleaving enzyme (i.e., spastin) onto MTs, leading to their rapid disassembly; HeLa cells co-transfected with the indicated constructs were treated with rapamycin (100 nM); scale bar, 10 µm. FIG. 2G shows recruitment of the artificially engineered microtubule-cleaving enzyme (i.e., spastin) onto MTs leads to their rapid disassembly, in which the normalized MT filament area in cells with different MT disruption treatments; n = 30, 45, 19, 20, 14, and 6 cells in nocodazole (3.3 µM), colchicine (500 µM), dNSpastin-C2Lact, dNSpastin3Q, dNSpastin3QED (SEQ ID NO:7), and dNSpastin3Q with MG132 (an effective, reversible and cell-permeable proteasome inhibitor) (50 µM) pre-treatment, respectively, from three independent experiments; data are shown as the mean ± S.E.M. FIGS. 2H to 2J show the MT disruption rates of MT-targeting agents (MTAs) and the MT disruption system of the present invention. In FIG. 2H, HeLa cells transfected with the indicated constructs were treated with nocodazole (Noc)(3.3 µM, a type of MTA), colchicine (Col)(500 µM, a type of MTA), or rapamycin (Rapa)(100 nM); scale bar, 10 µm. FIG. 2I shows the half time of MT disassembly triggered by the indicated MT disruption systems; n = 30, 45, 19, 30, and 6 cells from left to right, three to five independent experiments. FIG. 2J shows the relative proportion of MT area left in transfected HeLa cells after 1 hr of different MT-disrupting treatments. n = 34, 30, 45, 19, 20, 6, and 14 cells from left to right, three to five independent experiments. Data are shown as the mean ± S.E.M. Student’s t-tests were performed, with the resulting p-values indicated. Moreover, recruitment of dNSpastin3Q-YFP-FKBP onto MTs significantly removed more MT filaments as compared with others treatments (see FIGS. 2H and 2J). Trapping the enzyme-inactive dNSpastin3QED-YFP-FKBP onto MTs showed no MT disruption, indicating that acute MT disruption is an enzyme-dependent event (see FIGS. 2G to 2J).

This inducible MT disruption occurs in other cell types (see FIGS. 2K to 2N) and can also be controlled by rapamycin-orthogonal systems such as a gibberellin-based system with similar MT disruption kinetics (see FIGS. 2O to 2R). FIGS. 2K and 2L show rapid disruption of MTs in COS7 cells. In FIG. 2K, COS7 cells transfected with dNSpastin3Q-YFP-FKBP (green) and EMTB—CFP—FRB (blue) were treated with rapamycin (100 nM) and imaged; scale bar, 10 µm. Merge refers to the merging of fluorescent images. FIG. 2L shows the normalized MT filament area in cells transfected and treated as in FIG. 2K; n = 12 cells from four independent experiments. Data are shown as the mean ± S.E.M. FIGS. 2M and 2N show rapid disruption of MTs in U2OS cells. In FIG. 2M, U2OS cells transfected with dNSpastin3Q-YFP-FKBP (green) and EMTB—CFP—FRB (blue) were treated with rapamycin (100 nM) and imaged; scale bar, 10 µm. FIG. 2N shows rapid disruption of MTs in U2OS cells, in which the normalized MT filament area in cells transfected and treated as in FIG. 2M; n = 16 cells from four independent experiments. Data are shown as the mean ± S.E.M.

FIGS. 2O and 2P show using a gibberellin-based system to rapidly translocate proteins of interest onto MTs. In FIG. 2O, HeLa cells co-transfected with EMTB-CFP-mammalian optimized Gibberellin insensitive dwarf1 (mGID1)(blue)(the amino acid sequence of mGID1 is SEQ ID NO:13) and YFP-Gibberellin insensitive protein (GAIs)(SEQ ID NO:12) (green) (mGID1 and GAIs are dimerizing partners of the gibberellin system) were treated with GA3-AM (chemical dimerizer of the gibberellin system; 100 µM); live-cell imaging was used to monitor the FRET/CFP signal in cells; scale bar, 10 µm. FIG. 2P shows using a gibberellin-based system to rapidly translocate proteins of interest onto MTs, in which the normalized ratio of FRET/CFP in cells upon GA3-AM (blue) and 0.1% DMSO (red) treatment; n = 10 and 6 cells for the DMSO and GA3-AM treatments, respectively, from three independent experiments. Data are shown as the mean ± S.E.M. FIGS. 2Q and 2R show using the gibberellin-based system to rapidly disassemble MTs. In FIG. 2Q, HeLa cells co-transfected with dNSpastin3Q-YFP-GAIs (green) and EMTB—CFP—mGID1 (black and blue) were treated with GA3-AM (100 µM); scale bar, 10 µm. FIG. 2R shows using the gibberellin-based system to rapidly disassemble MTs, in which the normalized MT filament area in cells transfected with the constructs shown in FIG. 2Q upon GA3-AM treatment; n = 8 cells from three independent experiments. Data are shown as the mean ± S.E.M.

Moreover, spastazoline, a spastin inhibitor, swiftly reversed rapamycin-mediated acute MT disruption (see FIGS. 2S to 2U). FIGS. 2S to 2U show inhibition of spastin activity reverses MT disruption. In FIG. 2S, HeLa cells co-transfected with EMTB—CFP—FRB (black) and dNSpastin3Q-YFP-FKBP (green) were pre-treated with rapamycin for 28 min to induce acute MT disassembly; spastazoline (10 µM), a spastin inhibitor, was then added to the cultures to halt the MT disruption system; arrows indicate the centrosome-derived MTs; scale bar, 10 µm. FIG. 2T shows inhibition of spastin activity reverses MT disruption, in which normalized MT filament area for cells co-transfected and treated as in FIG. 2S; n = 3 cells from two independent experiments. Data are shown as the mean ± S.E.M. FIG. 2U shows inhibition of spastin activity reverses MT disruption, in which the polymerization rates of MTs regrowing from spastin-digested MT fragments in cytosol (acentrosome) and centrosomes; n = 147 and 74 MTs in the cytosol and centrosome group, respectively. The X-axis shows the growth rate (Y-axis) of microtubules from acentrosome and centrosome groups. Data (red) are shown as the mean ± SD. A Student’s t-test was performed to generate the indicated p-value.

Intriguingly, nascent MTs from the fragments derived from dNspastin3Q-mediated cleavage assembled at a polymerization rate of 0.92 ± 0.04 µm/sec, which was slightly slower than the regrowth rate of 1.17 ± 0.07 µm/sec for MTs from centrosomes (see FIG. 2U). Taken together, these results demonstrate that acute MT disruption occurs through MT filament disassembly instead of MT degradation. In particular, microtubule filaments are composed of microtubule molecules. If they are only disassembled into microtubule molecules and the raw materials are still there, they would have the opportunity to quickly assemble them back. But if the microtubule molecules are degraded and the raw materials are gone, they can’t be quickly reassembled. In summary, rapid recruitment of dNSpastin3Q-YFP-FKBP onto MTs efficiently triggered MT disassembly in living cells upon dimerization induction.

### Example 3

### Acute MT Disassembly Inhibits Vesicular Trafficking and Lysosome Dynamics

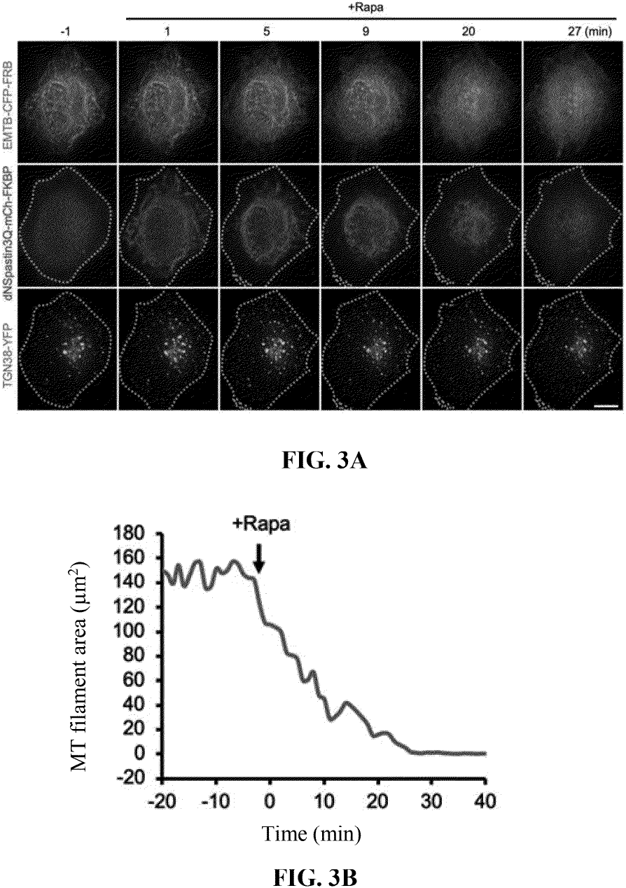

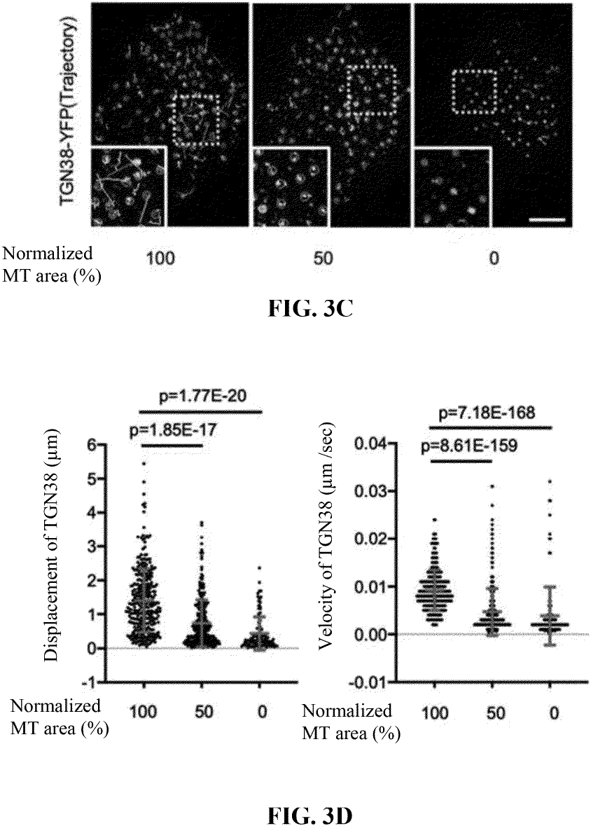

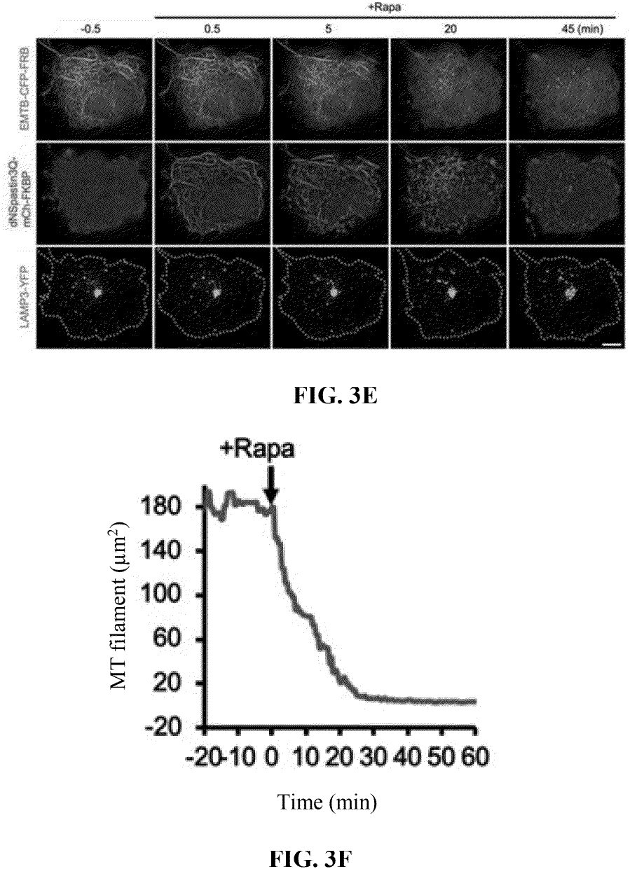

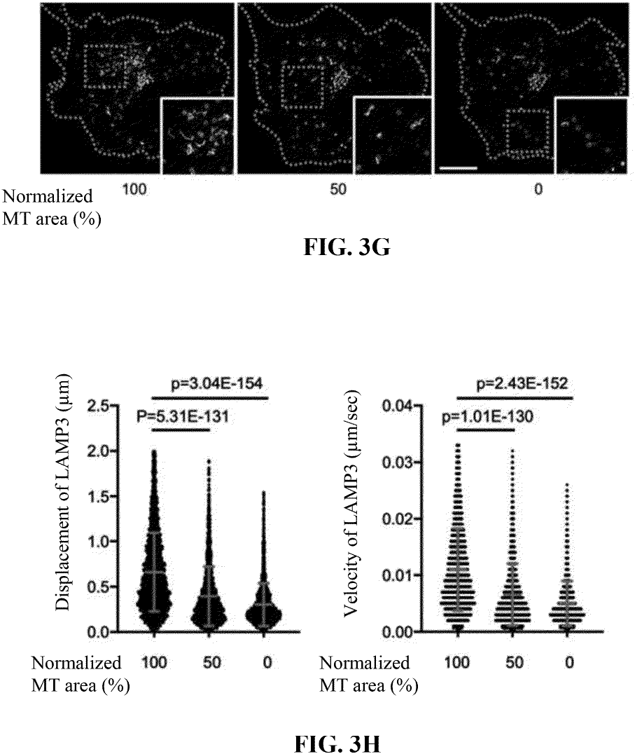

The inventors next evaluated whether acute MT disassembly functionally perturbs vesicle and organelle dynamics. To address this, a YFP-labeled post-Golgi vesicle marker, TGN38 (Trans-Golgi network integral membrane protein 38), and lysosome marker, LAMP3 (Lysosome-associated membrane glycoprotein 3), were used to observe the real-time dynamics of vesicles and lysosomes upon acute MT disassembly, respectively (see FIGS. 3A to 3H).

FIGS. 3A to 3D show acute MT disassembly attenuates vesicular trafficking. In FIG. 3A, HeLa cells co-transfected with EMTB—CFP—FRB (blue), dNSpastin3Q-mCherry-FKBP (dNSpastin3Q-mCh—FKBP) (red), and trans-Golgi network integral membrane protein 38-YFP (TGN38-YFP) (green) were treated with rapamycin (100 nM) to induce MT disruption; dotted lines indicate the cell boundary; scale bar, 10 µm. FIG. 3B shows acute MT disassembly attenuates vesicular trafficking, in which the MT filament area in the cell shown in FIG. 3A. FIG. 3C shows acute MT disassembly attenuates vesicular trafficking, in which trajectories of each TGN38-YFP-labeled vesicle at different levels of MT disruption; insets show higher-magnification images of the regions indicated by the dotted-line boxes; scale bar, 10 µm. FIG. 3D shows the displacement (left) and velocity (right) of each labeled vesicle shown in FIG. 3C; n = 312, 319, and 111 vesicles in the MT 100, 50, and 0% group, respectively. Data (red) are shown as the mean ± SD. Student’s t-tests were performed to generate the indicated p-values.

FIGS. 3E to 3H show acute MT disassembly attenuates lysosome dynamics. In FIG. 3E, HeLa cells co-transfected with EMTB—CFP—FRB (blue), dNSpastin3Q-mCh—FKBP (red), and LAMP3-YFP (green) were treated with rapamycin (100 nM) to induce MT disruption; dotted lines indicate the cell boundary; scale bar, 10 µm. FIG. 3F shows the MT filament area in the cell shown in FIG. 3E. FIG. 3G shows acute MT disassembly attenuates lysosome dynamics, in which trajectories of each LAMP3-YFP-labeled lysosome at different levels of MT disruption; insets show higher-magnification images of the regions indicated by the dotted-line boxes; scale bar, 10 µm. FIG. 3H shows displacement (left) and velocity (right) of each labeled lysosome shown in FIG. 3G; n = 2893, 2618, and 2315 lysosomes in the MT 100, 50, and 0% group, respectively. Data (red) are shown as the mean ± SD. Student’s t-tests were performed to generate the indicated p-values.

Acute MT disassembly attenuated the movement of post-Golgi vesicles and lysosomes in terms of moving distance and speed (see FIGS. 3A to 3H). Thus the claimed method not only disrupts MT structure but also rapidly blocks vesicular trafficking and lysosome dynamics.

### Example 4

### Rapid Disruption of Tyrosinated MTs

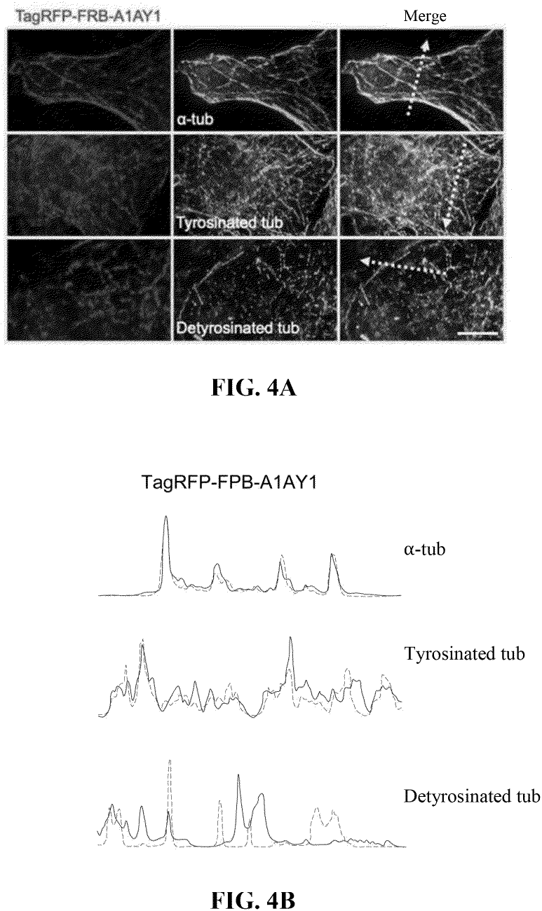

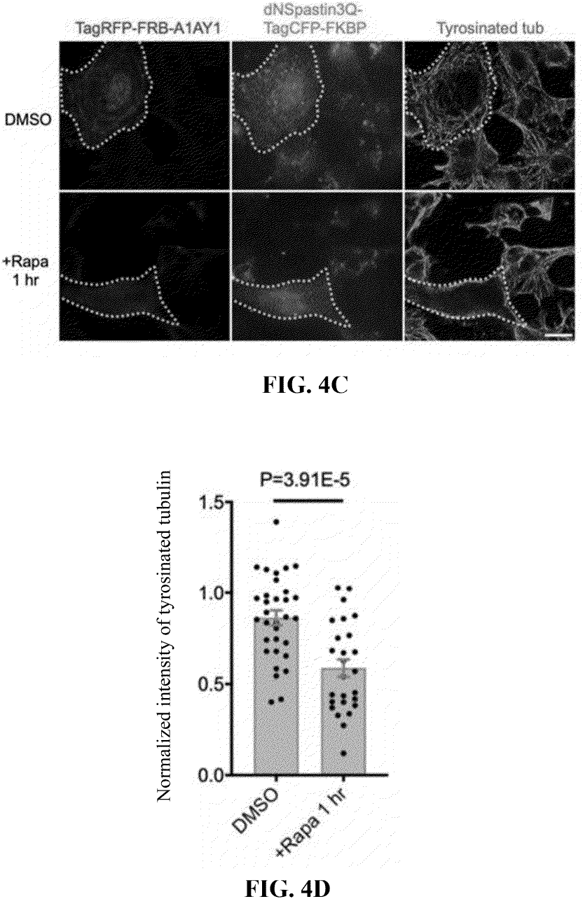

MTs undergo various post-translational modification (PTMs) for spatiotemporally regulating their properties and functions. The inventors next turned efforts to precisely disrupt MTs with specific PTMs. Very recently, an elegant work has established a protein, A1AY1 (SEQ ID NO:16), that specifically target to tyrosinated MTs in living cells. To apply it in the claimed invention, A1AY1 was tagged with FRB and a red fluorescent protein, TagRFP. Immunostaining results confirmed that the regulating construct (TagRFP—FRB—A1AY1) preferentially targeted to generic MTs and tyrosinated MTs but not to detyrosinated MTs (see FIGS. 4A and 4B).

FIGS. 4A and 4B show disruption of tyrosinated MTs. In FIG. 4A, COS7 cells transfected with TagRFP—FRB—A1AY1 (red) were labeled by anti-α-tubulin antibody (green; upper panel), anti-tyrosinated tubulin antibody (green; middle panel), or anti-detyrosinated tubulin antibody (green; lower panel), respectively. FIG. 4B shows disruption of tyrosinated MTs, in which the intensity profiles of TagRFP-FRB-A1AY1 (red) and tubulin with the indicated PTMs (green) along dotted lines drawn in FIG. 4A; the solid line represents TagRFP—FPB—A1AY1; the dotted lines from top to bottom indicate α-tubulin, tyrosinated tubulin, and detyrosinated tubulin, respectively. The addition of rapamycin rapidly recruited a cyan fluorescent protein, TagCFP, tagged FKBP (TagCFP—FKBP), onto TagRFP—FRB—A1AY1—bound MTs (T_{½} = 64.71 ± 16.08 sec). Moreover, recruitment of dNSpastin3Q—TagCFP—FKBP onto A1AY1-labeled MTs disrupted the A1AY1-positive MT filaments and significantly reduce about 41.4% of tyrosinated MTs (see FIGS. 4C and 4D). FIGS. 4C and 4D show disruption of tyrosinated MTs. In FIG. 4C, COS7 cells co-transfected with TagRFP—FRB—A1AY1 (red) and dNSpastin3Q—TagCFP—FKBP (cyan) were treated with 0.1% DMSO or rapamycin (100 nM) for 1 hr and following by immunostaining with anti-tyrosinated tubulin antibody (green); dotted lines highlight the transfected cells; scale bar, 20 µm. FIG. 4D shows disruption of tyrosinated MTs, in which the normalized intensity of tyrosinated MTs in TagRFP—FRB—A1AY1 and dNSpastin3Q-TagCFP-FKBP co-transfected cells upon 0.1% DMSO or rapamycin treatment for 1 hr; n=31 and 26 cells in DMSO and rapamycin treated groups, respectively, from three independent experiments. Data (blue) represent as mean ± S.E.M. Student’s t tests were performed with p values indicated. These results indicate that with specific tubulin PTMs biosensor against tyrosinated MTs, the claimed method is able to precisely disassemble MTs modified with tyrosination.

### Example 5

### Rapid Disruption of Specific MT-Based Structures

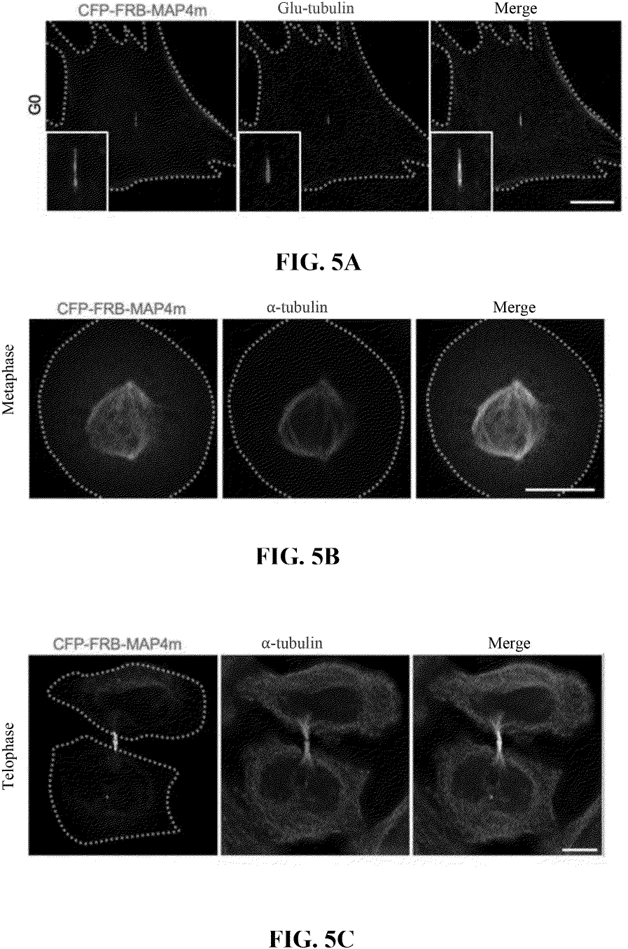

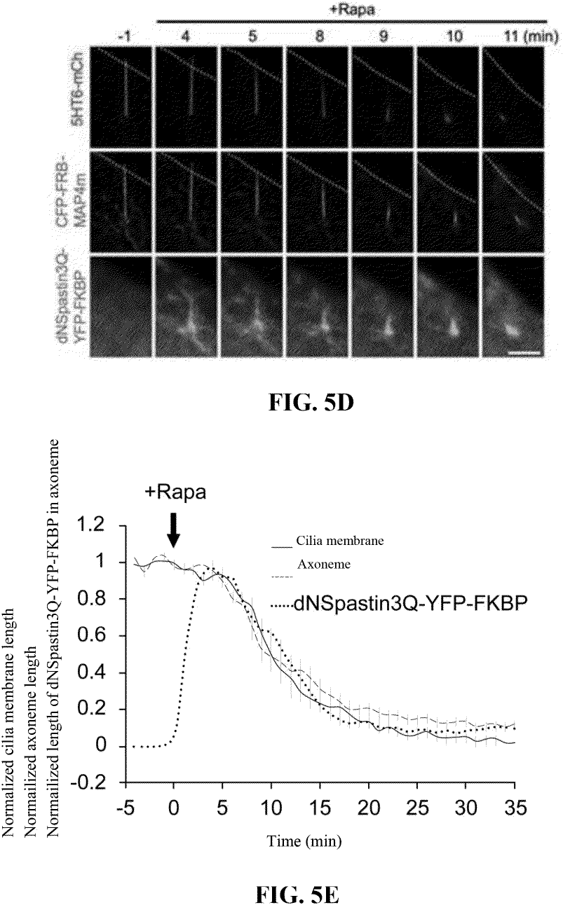

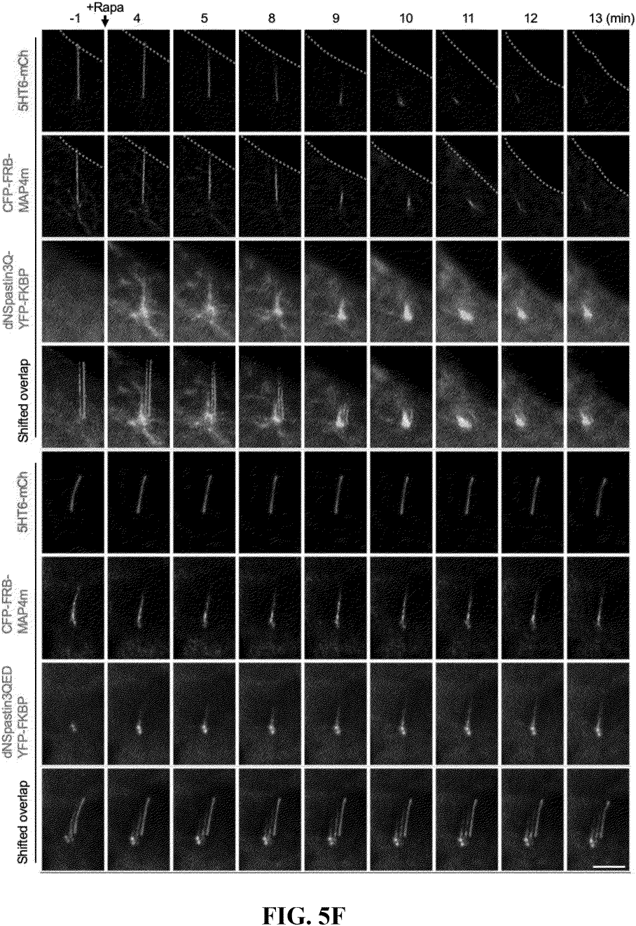

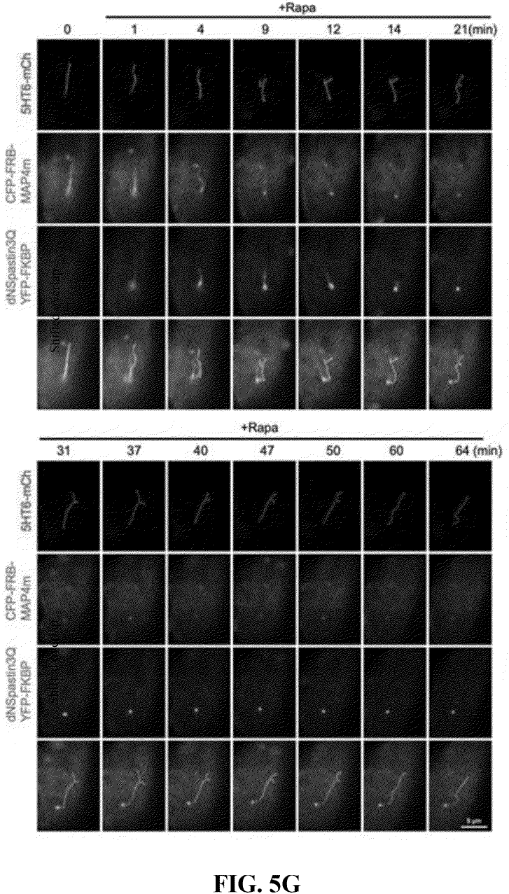

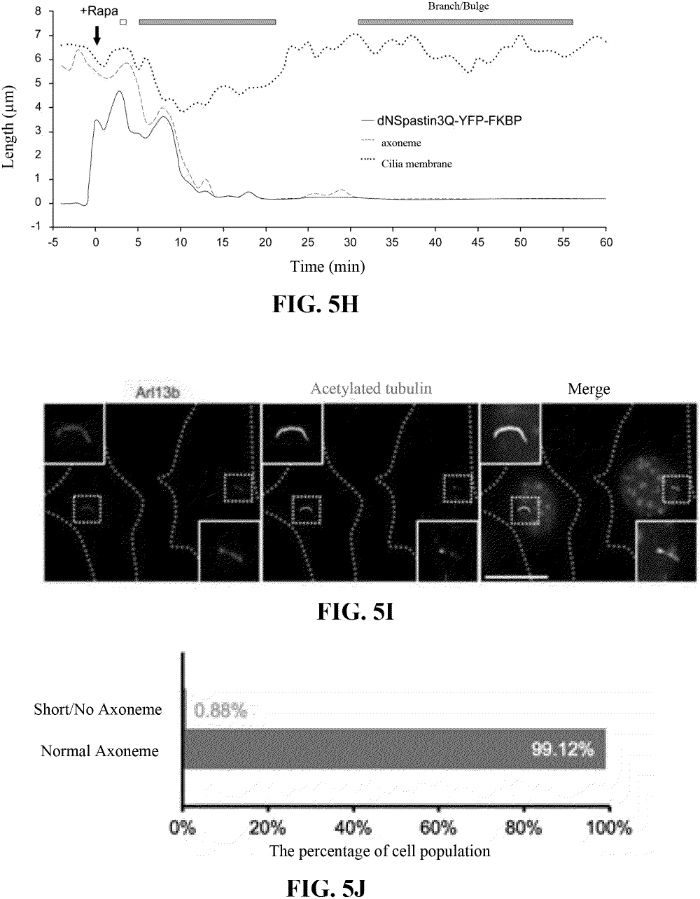

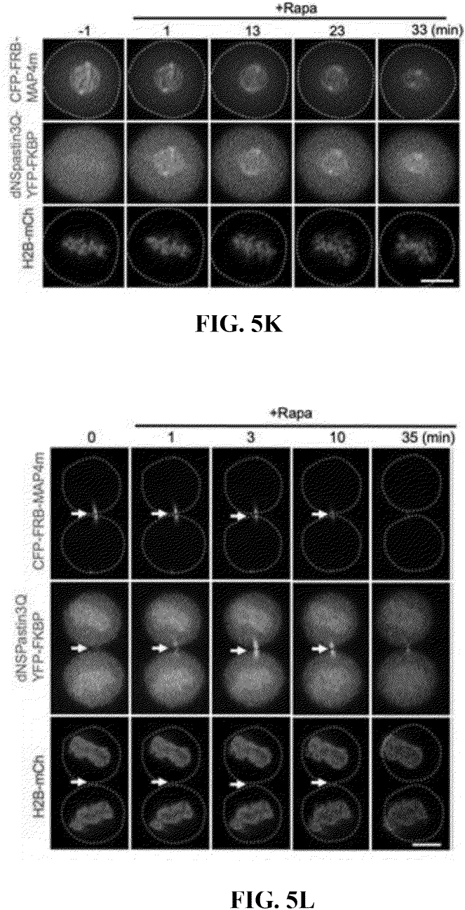

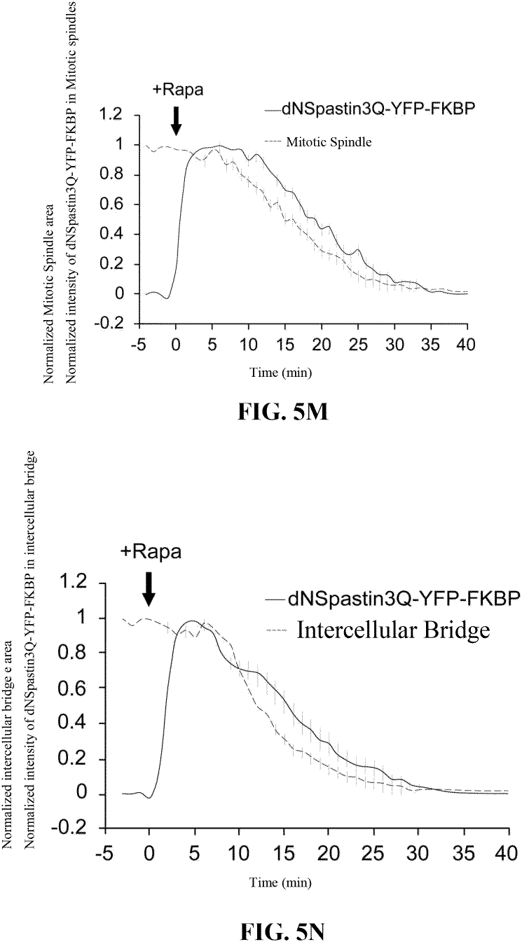

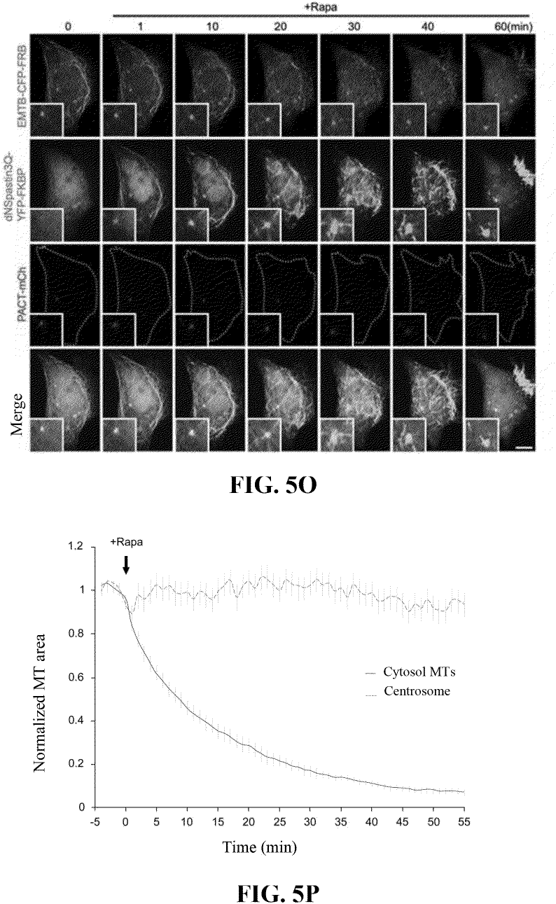

In addition to cytosol MTs, many MT-based structures including primary cilia, centrosomes, mitotic spindles, and intercellular bridges regulate various cellular activities in a defined spatiotemporal manner. The inventors next tested whether recruitment of dNSpastin3Q onto these different MT-based structures can specifically disassemble them. A truncated MT-binding domain of microtubule associate protein 4 (MAP4m)(SEQ ID NO:19) preferentially bound to ciliary axonemes in G0 cells and shifts to mitotic spindles during metaphase and intercellular bridges during telophase (see FIGS. 5A to 5C).

FIGS. 5A to 5C show subcellular distribution of CFP—FRB—MAP4m at different phases of the cell cycle. In FIG. 5A, NIH3T3 cells transfected with CFP—FRB—MAP4m (green) were serum starved to promote ciliogenesis; ciliated cells were fixed and labeled with GT335, an antibody that is specific for glutamylated tubulin (Glu-tub; red) at ciliary axonemes. FIGS. 5B and 5C show HeLa cells expressing CFP—FRB—MAP4m (green) were synchronized during metaphase (FIG. 5B) and telophase (FIG. 5C) and then were fixed and stained with α-tubulin antibody (red); dotted lines indicate cell boundaries; scale bar, 10 µm.

In G0 cells, addition of rapamycin rapidly trapped FKBP-tagged dNSpastin3Q on CFP—FRB—MAP4m—labeled axonemes in the cilia. Local accumulation of dNSpastin3Q on ciliary axonemes resulted in rapid disassembly of axonemes and primary cilia within 15 min (see FIGS. 5D to 5F).

FIGS. 5D and 5E show rapid disruption of primary cilia, mitotic spindles, and intercellular bridges. In FIG. 5D, NIH3T3 fibroblasts co-transfected with 5HT6-mCherry (5HT6-mCh; a ciliary membrane marker; red), CFP—FRB—MAP4m (blue), and dNSpastin3Q-YFP-FKBP (green) were serum starved for 24 hr to induce ciliogenesis; ciliated cells were then treated with rapamycin (100 nM) to induce dNSpastin3Q-YFP-FKBP recruitment to axonemal MTs; scale bar, 5 µm. FIG. 5E shows the normalized length of axonemes (Axoneme), primary cilia (Cilia membrane), and dNSpastin3Q-YFP-FKBP in cilia (dNSpastin3Q-YFP-FKBP) upon rapamycin treatment; n = 6 cells from three independent experiments.

FIG. 5F shows rapid disassembly of primary cilia, in which NIH3T3 cells co-transfected with 5HT6-mCh (a ciliary membrane marker; red), CFP—FRB—MAP4m (an axoneme marker; blue), and dNSpastin3Q-YFP-FKBP (upper panel; green) or enzyme dead dNSpastin3QED-YFP-FKBP (lower panel; green) were serum starved for 24 hr to induce ciliogenesis; ciliated cells were treated with rapamycin (100 nM) to recruit dNSpastin proteins onto axonemes; the recruitment of spastin proteins, morphology of ciliary axonemes, and ciliary membrane, upon rapamycin treatment was monitored by live-cell imaging; dotted lines indicate the cell boundary; scale bar, 5 µm.

Trapping enzyme-inactive dNSpastin3QED-YFP-FKBP on ciliary axonemes did not perturb the ciliary structure (see FIG. 5F). Although axonemes are known as major skeletons of primary cilia, acute axoneme disruption was occasionally not associated with disassembly of the entire ciliary structure and induced bulging and branched phenotypes in cilia (see FIGS. 5G and 5H).

FIGS. 5G and 5H show that the structure of the cilia occasionally does not collapse after axoneme disruption. In FIG. 5G, NIH3T3 cells co-transfected with 5HT6-mCh (ciliary membrane; red), CFP—FRB—MAP4m (axonemal MTs; blue), and dNSpastin3Q-YFP-FKBP (green) were serum starved to induce ciliogenesis; ciliated cells were treated with rapamycin (100 nM) to recruit dNSpastin3Q-YFP-FKBP onto axonemes; the recruitment of spastin proteins, morphology of the axonemes, and ciliary membrane were monitored upon rapamycin addition by live-cell imaging; scale bar, 5 µm. FIG. 5H shows the length of the indicated structures in the cell shown in FIG. 5G was measured and plotted; the time period during which the ciliary membrane became bulged and/or branched is shown with the gray lines above the graph.

The above results indicate that other MT-independent factors may also contribute to the maintenance of ciliary structures. This may explain why rare cells paradoxically showed a ciliary membrane that did not have axonemes (see FIGS. 5I and 5J).

FIGS. 5I and 5J show cells can spontaneously form ciliary membranes without axonemes. In FIG. 5I, NIH3T3 cells were serum starved to promote ciliogenesis; ciliated cells were fixed and labeled with antibodies against Arl13b (a ciliary membrane marker; red) and against acetylated tubulin (acetylated tub; a ciliary axoneme marker; green); dotted lines indicate cell boundaries; insets show higher-magnification images of the regions indicated by the dotted-line boxes; scale bar, 10 µm. FIG. 5J shows the percentage of cells that spontaneously formed ciliary membranes with a short or absent axoneme or a normal axoneme; n = 391 cells.

FIGS. 5K to 5N show rapid disruption of mitotic spindles and intercellular bridges. In FIG. 5K, HeLa cells co-transfected with H2B—mCherry (H2B—mCh; a chromosome marker; red), CFP—FRB—MAP4m (a marker of mitotic spindles; blue), and dNSpastin3Q-YFP-FKBP (green) were synchronized in metaphase and treated with rapamycin (100 nM) to induce dNSpastin3Q-YFP-FKBP recruitment to mitotic spindles; scale bar, 10 µm. In FIG. 5L, HeLa cells co-transfected with H2B—mCherry (red), CFP—FRB—MAP4m (intercellular bridges; blue), and dNSpastin3Q-YFP-FKBP (green) were treated with rapamycin (100 nM) to induce dNSpastin3Q-YFP-FKBP recruitment to intercellular bridges; arrows indicate the intercellular bridges; scale bar, 10 µm. FIG. 5M shows the normalized area of the mitotic spindle and intensity of dNSpastin3Q-YFP-FKBP in the mitotic spindle upon rapamycin treatment. n = 6 cells from three independent experiments. FIG. 5N shows the normalized area of intercellular bridges and intensity of dNSpastin3Q—YFP—FKBP at intercellular bridges upon rapamycin treatment. n = 5 cells from three independent experiments. Data are shown as the mean ± S.E.M. Dotted lines indicate the cell boundaries.

In mitotic cells, translocation of dNSpastin3Q onto CFP—FRB—MAP4m-labeled mitotic spindles (see FIG. 5K) and intercellular bridges (see FIG. 5L) quickly disrupted these MT-based structures (see FIGS. 5K to 5N).

Surprisingly, dNSpastin3Q did not disrupt centrosomes (or basal bodies in G0 cells) as shown by the images of cells that retained PACT (a conserved centrosomal targeting motif of pericentrin protein)-labeled centrosomes after MT disruption treatment for 1 hr (see FIGS. 5O and 5P).

FIGS. 5O and 5P show spastin does not disrupt centrosomes. In FIG. 5O, HeLa cells co-transfected with EMTB—CFP—FRB (blue), dNSpastin3Q-YFP-FKBP (green), and PACT-mCh (a centrosomal marker; red) were treated with rapamycin (100 nM) and imaged; dotted lines indicate the cell boundary; insets show higher-magnification images of the centrosome regions; scale bar, 10 µm. FIG. 5P shows the normalized area of MT filaments in the cytosol (Cytosol MTs) and centrosomes is shown; n = 8 cells from four independent experiments. Data are shown as the mean ± S.E.M.

### Example 6

### Using Light to Spatiotemporally Disrupt MTs

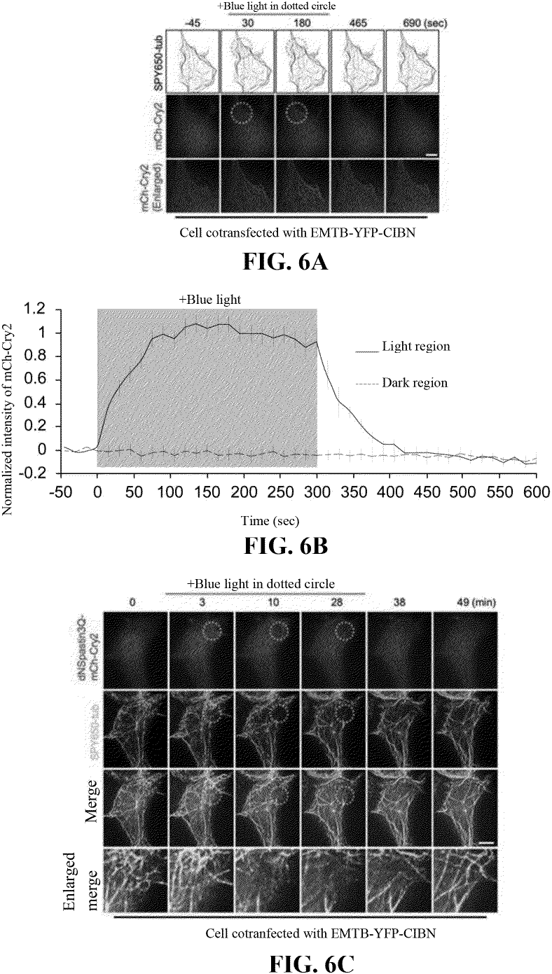

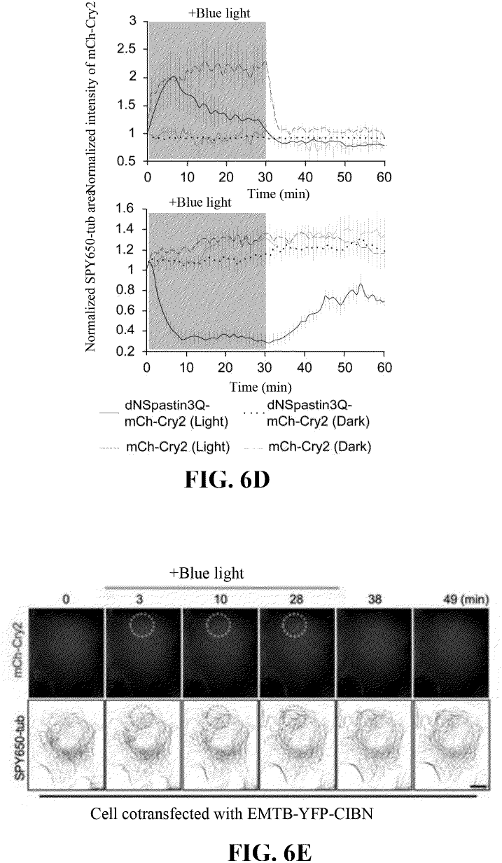

The inventors next tried to use an optogenetic system to trigger MT disassembly in a sub-cellular region of interest during a specific period of time. Cryptochrome 2 (Cry2) (SEQ ID NO:14) and CIBN (N-terminal 170 amino acids of CIB1) (SEQ ID NO:15), two blue light-sensitive dimerizing partners, were used in our MT-manipulating system. Cry2, which was tagged with the red fluorescent protein mCherry (mCh—Cry2), was rapidly translocated onto EMTB—YFP—CIBN-labeled MTs only in regions illuminated with light (T_{½} = 34.6 ± 7.58 sec) and dissociated from MTs to the cytosol when the light was off (T_{½} = 28.2 ± 4.88 sec; see FIGS. 6A and 6B).

FIGS. 6A and 6B show using light to disassemble MTs in a reversible and location-specific manner. In FIG. 6A, COS7 cells co-transfected with EMTB—YFP—CIBN and mCh—Cry2 (red) were incubated with SPY650-tubulin (SPY650-tub) to visualize MTs; the cells were illuminated by blue light within a specified region (indicated by the dotted circle) for the indicated time period; scale bar, 10 µm. FIG. 6B shows the normalized intensity of mCh—Cry2 at MTs in illuminated regions (Light region) and non-illuminated regions (Dark region); n = 6 cells from three independent experiments.

The inventors then used light to control spastin-mediated MT disassembly in a spatially and temporally specific manner. dNSpastin3Q tagged with mCh-Cry2 was co-expressed with EMTB—YFP—CIBN in COS7 cells. MTs were labeled with SPY650-tubulin in these experiments. Local illumination with blue light robustly recruited dNSpastin3Q-mCh—Cry2 onto MTs, leading to the disassembly of MTs only in illuminated regions. The dNSpastin3Q-mCh—Cry2 reverted to cytosol and MT reassembly occurred when the cells were placed back in the dark (see FIGS. 6C and 6D).

FIGS. 6C and 6D show using light to disassemble MTs in a reversible and location-specific manner. In FIG. 6C, COS7 cells co-transfected with EMTB-YFP-CIBN and dNSpastin3Q-mCh—Cry2 were incubated with SPY650-tubulin to visualize MTs; the cells were illuminated by blue light as in FIG. 6A; scale bar, 10 µm. FIG. 6D shows the normalized intensity of dNSpastin3Q-mCh—Cry2 and mCh—Cry2 at MTs and the normalized area of SPY650-tubulin in illuminated regions and non-illuminated regions; n = 6 and 6 cells in dNSpastin3Q-mCh—Cry2 and mCh—Cry2 group, respectively, from three independent experiments. Data are shown as the mean ± S.E.M.

Recruitment of mCh—Cry2 without Spastin onto MTs by the same photostimulation procedure did not lead to MT disassembly, confirming that light-induced MT disassembly did not result from phototoxicity (see FIG. 6E).

FIG. 6E shows recruitment of light-sensitive dimerizing proteins without spastin does not disrupt MTs, in which COS7 cells were co-transfected with EMTB—YFP—CIBN and mCh—Cry2 (red), and MTs were labeled with SPY650-tubulin (SPY650-tub; black); illumination with blue light for the indicated period of time triggered rapid recruitment of mCh—Cry2 onto MTs in the light-illuminated region (dotted circle); the MTs, however, remained intact after recruitment of mCh—Cry2; scale bar, 10 µm.

In summary, the method and the platform for disrupting intracellular microtubules can accurately and quickly disrupt the microtubule structures in specific areas of cells by adding specific chemical components or specific wavelengths of light. In addition to providing important reagents for microtubule-related research in basic science, it may even be developed into a new technology for precise chemotherapy. Compared with two common traditional microtubule-binding drugs, nocodazole and colchicine, the present invention can disrupt intracellular microtubules faster (8.53 to 9.67 times faster), and remove more intracellular microtubules within one hour (3.3 µM nocodazole removes 31% of microtubules; 500 µM colchicine removes 49% of microtubules; the present invention removes 93% of microtubules). Also, the present invention can precisely disrupt specific microtubule structures, such as primary cilia, mitotic spindle and intercellular bridge, and solve the technical limitation that traditional microtubule binding drugs cannot specifically disrupt microtubule structures.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for disrupting intracellular microtubules, comprising the following steps:
(a) expressing an artificially engineered microtubule-cleaving enzyme on a cytosol, and expressing a microtubule-binding protein on the intracellular microtubules; and
(b) driving the artificially engineered microtubule-cleaving enzyme to the intracellular microtubules to dimerize with the microtubule-binding protein by using a chemical component, thereby allowing the artificially engineered microtubule-cleaving enzyme to recruit onto the intracellular microtubules and specifically disrupt a structure of the intracellular microtubules.

2. The method according to claim 1, wherein the chemical component is a macrolide compound or a tetracyclic diterpene compound.

3. The method according to claim 2, wherein the macrolide compound is rapamycin.

4. The method according to claim 3, wherein when the chemical component is rapamycin, the artificially engineered microtubule-cleaving enzyme comprises FK506-binding protein (FKBP).

5. The method according to claim 4, wherein when the chemical component is rapamycin, the microtubule-binding protein is FKBP-rapamycin binding domain (FRB).

6. The method according to claim 2, wherein the tetracyclic diterpene compound is gibberellin.

7. The method according to claim 6, wherein when the chemical component is gibberellin, the microtubule-binding protein is Gibberellin insensitive protein (GAIs).

8. The method according to claim 7, wherein when the chemical component is gibberellin, the artificially engineered microtubule-cleaving enzyme comprises mammalian optimized Gibberellin insensitive dwarf1 (mGID1).

9. The method according to claim 4, wherein the artificially engineered microtubule-cleaving enzyme further comprises an artificially engineered spastin.

10. The method according to claim 9, wherein three amino acid residues of the artificially engineered spastin are mutated into three consecutive glutamines by site-directed mutagenesis, and the artificially engineered spastin is a truncated spastin lacking 1st to 140th amino acids at N-terminus.

11. The method according to claim 8, wherein the artificially engineered microtubule-cleaving enzyme further comprises an artificially engineered spastin.

12. The method according to claim 11, wherein three amino acid residues of the artificially engineered spastin are mutated into three consecutive glutamines by site-directed mutagenesis, and the artificially engineered spastin is a truncated spastin lacking 1st to 140th amino acids at N-terminus.

13. The method according to claim 1, wherein the structure of the intracellular microtubules is primary cilia, mitotic spindle or intercellular bridge.

14. The method according to claim 13, wherein the primary cilia comprises an axoneme and a ciliary membrane, and when the primary cilia is disrupted, the ciliary membrane is in a bulging and branched phenotype.

15. The method according to claim 1, wherein the intracellular microtubules are disrupted within one hour.

16. The method according to claim 1, wherein the intracellular microtubules are disrupted in a reversible manner.

17. The method according to claim 5, wherein each of the intracellular microtubules is a tyrosinated microtubule.

18. The method according to claim 17, wherein the FRB tags an A1AY1 protein.

19. A method for disrupting intracellular microtubules, comprising the following steps:
(a) expressing an artificially engineered microtubule-cleaving enzyme on a cytosol, and expressing a plurality of microtubule-binding proteins on the intracellular microtubules; and
(b) using a light to stimulate the artificially engineered microtubule-cleaving enzyme and the plurality of microtubule-binding proteins,
wherein the light induces dimerization of the plurality of microtubule-binding proteins and the artificially engineered microtubule-cleaving enzyme, thereby allowing the artificially engineered microtubule-cleaving enzyme to recruit onto the intracellular microtubules and specifically disrupt a structure of the intracellular microtubules.

20. The method according to claim 19, wherein the light is blue light.

21. The method according to claim 19, wherein the plurality of microtubule-binding proteins are cryptochrome 2 (Cry2) and N-terminal 170 amino acids of calcium and integrin-binding protein 1 (C1B1)(CIBN).

22. The method according to claim 19, wherein the artificially engineered microtubule-cleaving enzyme comprises an artificially engineered spastin.

23. The method according to claim 19, wherein the structure of the intracellular microtubules is primary cilia, mitotic spindle or intercellular bridge.

24. The method according to claim 23, wherein the primary cilia comprises an axoneme and a ciliary membrane, and when the primary cilia is disrupted, the ciliary membrane is in a bulging and branched phenotype.

25. The method according to claim 19, wherein the intracellular microtubules are disrupted within one hour.

26. The method according to claim 19, wherein the artificially engineered microtubule-cleaving enzyme specifically disrupts the structure of the intracellular microtubules in an illuminated region.

27. The method according to claim 20, wherein the intracellular microtubules are disrupted in a reversible manner.

28. A platform for disrupting intracellular microtubules, being established by the method according to claim 1.
